(19) United States
(12) Patent Application Publication
(10) Pub. No.: US 2004/0077522 A1
(43) Pub. Date: Apr. 22, 2004

(54) Crystals and structure of luxs

(76) Inventor: Hal A. Lewis, San Diego, CA (US)

Correspondence Address: JONES DAY, 222 EAST 41ST STREET, NEW YORK, NY, 10017, US

(21) Appl. No.: 10/398,424

(22) PCT Filed: Oct. 1, 2001
(86) PCT No.: PCT/US01/30684

(30) Foreign Application Priority Data
Dec. 4, 2000 (US) 09/729838

(51) Int. Cl. A61K 31/00; G06F 19/00; G01N 33/48; G01N 33/50

(52) U.S. Cl. 514/1; 702/19

(57) ABSTRACT

The present invention provides crystalline LuxS, machine readable media embedded with the three-dimensional atomic structure coordinates of LuxS, and subsets thereof, and methods of using them.

### 1. CROSS REFERENCE TO RELATED APPLICATIONS

This application claims the benefit of priority of provisional application Serial No. 60/237,933, filed Oct. 3, 2000, and U.S. utility application Ser. No. 09/729,838, filed Dec. 4, 2000, the contents of which are hereby incorporated by reference.

### 2. FIELD OF THE INVENTION

The present invention concerns crystalline forms of polypeptides that correspond to LuxS, methods of obtaining such crystals and the high-resolution X-ray diffraction structures and atomic structure coordinates obtained therefrom. The crystals of the invention and the atomic structural information obtained therefrom are useful for solving the crystal and solution structures of related and unrelated LuxSs, and for screening for, identifying and/or designing compounds that bind and/or modulate a biological activity of LuxS. The atomic structural information may also be used to design novel mutant forms of LuxS polypeptides.

### 3. BACKGROUND OF THE INVENTION

LuxS protein is involved in the production of autoinducer-2 (AI-2), an intercellular signaling molecule employed in the quorum sensing pathway of various bacteria (WO 00/32152). These bacteria include pathogens such as Helicobacter pylori, Haemophilus influenzae, Campylorbacter jejuni, Salmonella typhimurium, Vibrio cholerae and Nesseria meningitidis.

Intercellular communication between bacteria regulates several key bacterial functions, such as competence and sporulation in Bacillus, sporulation and motility in Myxococcus and quorum sensing in bacteria such as luminous Vibrio. Each communication system uses a different small extracellular signaling molecule, all of which are amino acid based. For instance, such signaling molecules include acyl-homoserine lactones (HSL), peptides, and a mixture of amino acids and fragments of peptidoglycan, respectively. V. harveyi uses two independent cell-cell communication systems in controlling its luminescence expression. Signaling system 1 is highly species specific and uses a homoserine signal. System 2 is not as well characterized, but appears to not be species-specific. It has been proposed that it employs 4,5-dihydroxy-2,3-pentanedione as its signal (PCT WO 00/32152), and has also been proposed that the AI-2 signal is a furanone (Schauder, S. and Bassler, B. L., Genes and Development 15:1468-80, (2001)) and that its substrate is S-ribosylhomocysteine.

Quorum sensing bacteria synthesize, release and respond to specific autoinducers in order to control gene expression as a function of cell density. The quorum sensing pathways are important in the virulence of some bacteria. For example, Pseudomonas aeruginosa exists as biofilms in cystic fibrosis lungs. Quorum sensing via signaling system 1 is employed by P. aeruginosa to enable biofilm formation. Enterohemorrhagic E. coli (EHEC) is responsible for hemorrhagic colitis and hemolytic uremic syndrome. Enteropathogenic E. coli (EPEC) is responsible for infant diarrhea. Virulence in these E. coli bacteria is controlled by expression of the type III secretion system of which LuxS is a crucial component. LuxS thus provides a target for novel compounds that can be used to treat or prevent virulent infections of microorganisms such as EPEC. In addition, certain crop pests, for example, Erwinia carotovora, Ralstonia solanacearum, and Agrobacterium tumefaciens require an intact quorum-sensing pathway for pathogenicity.

LuxS thus also provides a target for novel compounds that can be used as pesticides or plant anti-microbials.

Until the present invention, the ability to obtain the atomic structure coordinates of LuxS has not been realized. Crystals of LuxS and the atomic structure coordinates of LuxS would enable further study of the LuxS protein. Significantly, the atomic structure coordinates of LuxS would enable the design and selection of antibiotics that target bacterial strains whose pathogenesis depends on the quorum sensing pathway.

### 4. SUMMARY OF THE INVENTION

In one aspect, the invention provides crystalline forms of polypeptides corresponding to LuxS of the LuxS Family type. The LuxS Family include proteins represented by SWISSPROT accession numbers Q9ZMW8, O24931, Q9XDU6, O34667, D75280, Q9Z5X1, P45578, P44007, O50164 and other related polypeptides. The crystals of the invention comprise crystallized polypeptides corresponding to the wild-type or mutated LuxS. The crystals of the invention include native crystals, in which the crystallized LuxS is substantially pure; heavy-atom derivative crystals, in which the crystallized LuxS is in association with one or more heavy-metal atoms; and co-crystals, in which the crystallized LuxS is in association with one or more compounds, including but not limited to, cofactors, ligands, substrates, substrate analogs, inhibitors, allosteric effectors, etc. to form a crystalline co-complex. Preferably, such compounds bind a catalytic or active site or a site on the LuxS molecule that modulates a biological activity of the LuxS protein. The co-crystals may be native co-crystals, in which the co-complex is substantially pure, or they may be heavy-atom derivative co-crystals, in which the co-complex is in association with one or more heavy-metal atoms.

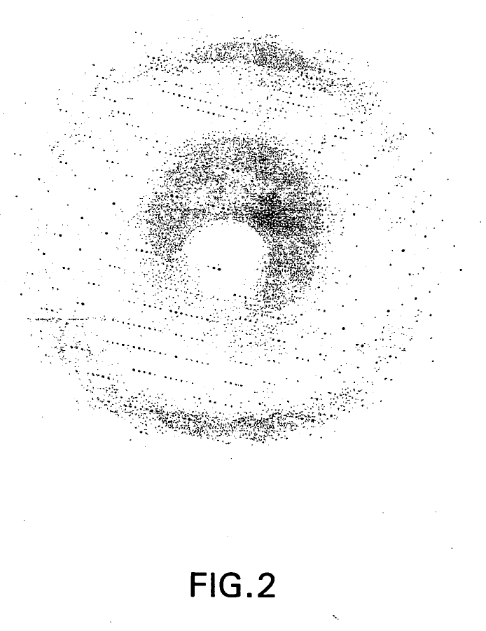

The Examples discuss crystals of the invention that were obtained for LuxS protein from three bacteria: Helicobacter pylori, Haemophilus influenzae, and Deinococcus radiodurans (two forms). They are generally characterized by a unit cell of a=71.04+/−1% Å, b=71.04+/−1% Å, c=130.14+/−1% Å, α=90, β=90, γ=90 for H. pylori); a=129.59+/−1% Å, b=129.59+/−1% Å, c=53.74+/−1% Å, α=90, β=90, γ=90 (for H. influenzae); a=43.71+/−1% Å, b=82.18+/−1% Å, c=49.48+/−1% Å, α=90, β=90, γ=90 (for D. radiodurans); and a=51.19+/−1% Å, b=70.14+/−1% Å, c=49.73+/−1% Å, α=90, β=112.03, γ=90 (also for D. radiodurans). The H. pylori protein grew crystals in space group P4₃2₁2. The H. influenzae protein grew crystals in space group P4₂2₁2. The D. radiodurans protein grew crystals in the P2₁, C2, and P1 space groups. These crystals are preferably of diffraction quality. Representatative diffraction images for the four LuxS crystals are shown in FIGS. 2-5. In more preferred embodiments, the crystals of the invention are of sufficient quality to permit the determination of the three-dimensional X-ray diffraction structure of the crystalline polypeptide to high resolution, preferably to a resolution of greater than about 3 Å, typically in the range of about 1 Å to about 3 Å or in the range of about 1.8 Å to about 2.4 Å.

The invention also provides methods of making the crystals of the invention. Generally, native crystals of the invention are grown by dissolving substantially pure polypeptide in an aqueous buffer that includes a precipitant at a concentration just below that necessary to precipitate the polypeptide. Water is then removed by controlled evaporation to produce precipitating conditions, which are maintained until crystal growth ceases.

Co-crystals of the invention are prepared by soaking a native crystal prepared according to the above method in a liquor comprising the compound of the desired co-complex. Alternatively, the co-crystals may be prepared by co-crystallizing the polypeptide in the presence of the compound according to the method discussed above.

Heavy-atom derivative crystals of the invention may be prepared by soaking native crystals or co-crystals prepared according to the above method in a liquor comprising a salt of a heavy atom or an organometallic compound. Alternatively, heavy-atom derivative crystals may be prepared by crystallizing a polypeptide comprising selenomethionine and/or selenocysteine residues according to the methods described previously for preparing native crystals.

In another aspect, the invention provides machine and/or computer-readable media embedded with the three-dimensional structural information obtained from the crystals of the invention, or portions or subsets thereof. Such three-dimensional structural information will typically include the atomic structure coordinates of the crystallized polypeptide or co-complex, or the atomic structure coordinates of a portion thereof such as, for example, the atomic structure coordinates of the amino acid residues corresponding to an active or binding site, but may include other structural information, such as vector representations of the atomic structures coordinates, etc. The types of machine- or computer-readable media onto which the structural information is embedded typically include magnetic tape, floppy discs, hard disc storage media, optical discs, CD-ROM, electrical storage media such as RAM or ROM, and hybrids of any of these storage media. Such media also include paper on which is recorded the structural information that can be read by a scanning device and converted into a three-dimensional structure with an OCR and further includes stereo diagrams of three-dimensional structures from which coordinates can be derived. The machine and/or computer-readable media of the invention may further comprise additional information that is useful for representing the three-dimensional structure of the crystalline polypeptides, including, but not limited to, thermal parameters, chain identifiers, and connectivity information.

The invention is illustrated by way of working examples demonstrating the crystallization and characterization of crystals, the collection of diffraction data, and the determination and analysis of the three-dimensional structures of LuxS protein from these different bacterial species: Helicobacter pylori, Haemophilus influenzae and Deinococcus radiodurans.

The atomic structure coordinates and machine readable media of the invention have a variety of uses. For example, the coordinates are useful for solving the three-dimensional X-ray diffraction and/or solution structures of other LuxSs, including mutant LuxS, co-complexes comprising LuxS, and unrelated LuxSs, to high resolution. Structural information may also be used in a variety of molecular modeling and computer-based screening applications to, for example, intelligently design mutants of the crystallized LuxS that have altered biological activity and to computationally design and identify compounds that bind the polypeptide or a portion or fragment of the polypeptide, such as the active site. Such compounds may be used as lead compounds in pharmaceutical efforts to identify compounds that inhibit LuxS as a therapeutic approach toward the treatment of, e.g., infectious disease (LuxS inhibitors may be good antibiotics), stomach cancer, stomach ulcers and other intestinal complications (Helicobacter pylori is a causative agent for stomach ulcers and stomach cancer). Such compounds can also be used to treat or prevent, for example, infectious disease caused by pathogens such as Haemophilus influenzae, Campylobacter jejuni, Salmonella typhimurium, Vibrio cholerae and Nesseria meningitidis.

In an embodiment of the present invention, a method is provided of producing a mutant of LuxS, having an altered property relative to LuxS, comprising, a) constructing a three-dimensional structure of LuxS having structure coordinates selected from the group consisting of the structure coordinates of the crystals of the present invention, the structure coordinates of Table 7, 8, 9, 10, 11, or 12, and the structure coordinates of a protein having a root mean square deviation of the alpha carbon atoms of the protein of up to about 2 Å, preferably up to about 1.75 Å, preferably up to about 1.5 Å, preferably up to about 1.0 Å, and preferably up to about 0.75 Å, when compared to the structure coordinates of Table 7, 8, 9, 10, 11, or 12; b) using modeling methods to identify in the three-dimensional structure at least one structural part of the LuxS molecule wherein an alteration in the structural part is predicted to result in the altered property; c) providing a nucleic acid molecule having a modified sequence that encodes a deletion, insertion, or substitution of one or more amino acids at a position corresponding to the structural part; and d) expressing the nucleic acid molecule to produce the mutant; wherein the mutant has at least one altered property relative to the parent. The mutant may, for example, have altered LuxS activity. The altered LuxS activity may be, for example, altered binding activity, altered enzymatic activity, and altered immunogenicity, such as, for example, where an epitope of the protein is altered because of the mutation. The mutation that alters the epitope may be, for example, within the region of the protein that comprises the epitope. Or, the mutation may be, for example, at a site outside of the epitope region, yet causes a conformational change in the epitope region. Those of ordinary skill in the art will recognize that the region that contains the epitope may comprise either contiguous or non-contiguous amino acids.

In another embodiment of the present invention, a method is provided of identifying a compound that potentially binds LuxS, comprising computationally screening a three-dimensional structural representation of LuxS or a portion thereof, or a molecule comprising a LuxS binding pocket or binding pocket homolog, with a plurality of chemical compounds and chemical entities.

In another embodiment of the present invention, a method is provided of selecting at least one compound that potentially binds to LuxS, comprising, constructing a three-dimensional structure of LuxS having structure coordinates selected from the group consisting of the structure coordinates of the crystals of the present invention, the structure coordinates of Table 7, 8, 9, 10, 11, or 12, and the structure coordinates of a protein having a root mean square deviation of the alpha carbon atoms of up to about 2.0 Å, preferably up to about 1.75 Å, preferably up to about 1.5 Å, preferably up to about 1.0 Å, and preferably up to about 0.75 Å, when compared to the structure coordinates of Table 7, 8, 9, 10, 11, or 12, or a portion thereof, or constructing a three-dimensional structure of a molecule comprising a LuxS binding pocket or binding pocket homolog; and selecting at least one compound which potentially binds LuxS; wherein the selecting is performed with the aid of the constructed structure.

It is anticipated that in some cases, upon binding a compound, the conformation of the protein may be altered. Useful compounds may bind to this altered conformational form. Thus, included within the scope of the present invention are methods of selecting compounds that potentially bind to a LuxS molecule or homolog where the molecule or homolog comprises an amino acid sequence that is at least 20%, preferably at least 25%, more preferably at least 30%, more preferably at least 40%, the amino acid sequence of FIG. 2, more preferably at least 50%, using, for example, a PSI BLAST search, for example, but not limited to version 2.1.2 (Altschul, S. F., et al., Nuc. Acids Rec. 25:3389-3402 (1997)). Preferably at least 50%, more preferably at least 70% of the sequence is aligned in this analysis and where at least 50%, more preferably 60%, more preferably 70%, more preferably 80%, and most preferably 90% of the amino acids of the molecule or homolog have structure coordinates selected from the group consisting of the structure coordinates of the crystals of the present invention, the structure coordinates of Table 7, 8, 9, 10, 11, or 12, and the structure coordinates of a protein having a root mean square deviation of the alpha carbon atoms of up to about 2.0 Å, preferably up to about 1.75 Å, preferably up to about 1.5 Å, preferably up to about 1.0 Å, and preferably up to about 0.75 Å, when compared to the structure coordinates of Table 7, 8, 9, 10, 11, or 12, or a portion thereof, or constructing a three-dimensional structure of a molecule comprising a LuxS binding pocket or binding pocket homolog; and selecting at least one compound which potentially binds LuxS; wherein the selecting is performed with the aid of the constructed structure.

Also provided in the present invention is a method of identifying at least one compound that potentially binds to LuxS, comprising, constructing a three-dimensional structure of a protein molecule comprising a LuxS binding pocket or binding pocket homolog, or constructing a three-dimensional structure of a molecule comprising a LuxS binding pocket, and computationally screening a plurality of compounds using the constructed structure, and identifying at least one compound that computationally binds to the structure. In a preferred aspect, the method further comprises determining whether the compound binds LuxS.

In yet another aspect of the present invention, a method is provided of identifying a modulator of LuxS by rational drug design, comprising; designing a potential modulator of LuxS that forms covalent or non-covalent bonds with amino acids in a binding pocket of LuxS based on the molecular structure coordinates of the crystals of the present invention, or based on the molecular structure coordinates of a molecule comprising a LuxS binding pocket or binding pocket homolog; synthesizing the modulator; and determining whether the potential modulator affects the activity of LuxS. Preferably, the binding pocket comprises the active site of LuxS. The binding pocket may instead comprise an allosteric binding site of LuxS. A modulator may be, for example, an inhibitor, an activator, or an allosteric modulator of LuxS.

Other methods of designing modulators of LuxS include, for example, a method for identifying a modulator of LuxS activity comprising: providing a computer modeling program with a three dimensional conformation for a molecule that comprises a binding pocket of LuxS, or binding pocket homolog; providing a said computer modeling program with a set of structure coordinates of a chemical entity; using said computer modeling program to evaluate the potential binding or interfering interactions between the chemical entity and said binding pocket, or binding pocket homolog; and determining whether said chemical entity potentially binds to or interferes with said molecule; wherein binding to the molecule is indicative of potential modulation, including, for example, inhibition of LuxS activity.

In another embodiment, a method is provided for designing a modulator of LuxS activity comprising: providing a computer modeling program with a set of structure coordinates, or a three dimensional conformation derived therefrom, for a molecule that comprises a binding pocket of LuxS, or binding pocket homolog; providing a said computer modeling program with a set of structure coordinates, or a three dimensional conformation derived therefrom, of a chemical entity; using said computer modeling program to evaluate the potential binding or interfering interactions between the chemical entity and said binding pocket, or binding pocket homolog; computationally modifying the structure coordinates or three dimensional conformation of said chemical entity; and determining whether said modified chemical entity potentially binds to or interferes with said molecule; wherein binding to the molecule is indicative of potential modulation of LuxS activity. In other preferred aspects, determining whether the chemical entity potentially binds to said molecule comprises performing a fitting operation between the chemical entity and a binding pocket, or binding pocket homolog, of the molecule or molecular complex; and computationally analyzing the results of the fitting operation to quantify the association between, or the interference with, the chemical entity and the binding pocket, or binding pocket homolog. In a further embodiment, the method further comprises screening a library of chemical entities.

The LuxS inhibitor may also be designed de novo. Thus, the present invention also provides a method for designing a modulator of LuxS, comprising: providing a computer modeling program with a set of structure coordinates, or a three dimensional conformation derived therefrom, for a molecule that comprises a binding pocket having the structure coordinates of the binding pocket of LuxS, or a binding pocket homolog; computationally building a chemical entity represented by set of structure coordinates; and determining whether the chemical entity is a modulator expected to bind to or interfere with the molecule wherein binding to the molecule is indicative of potential modulation of LuxS activity. In other preferred embodiments, determining whether the chemical entity potentially binds to said molecule comprises performing a fitting operation between the chemical entity and a binding pocket of the molecule or molecular complex, or a binding pocket homolog; and computationally analyzing the results of the fitting operation to quantify the association between, or the interference with, the chemical entity and the binding pocket, or a binding pocket homolog.

In yet other preferred embodiments, once a modulator is computationally designed or identified, the potential modulator may be supplied or synthesized, then assayed to determine whether it inhibits LuxS activity.

Thus, the present invention also provides modulators of LuxS activity identified, designed, or made according to any of the methods of the present invention, as well as pharmaceutical compositions comprising such modulators. Preferred pharmaceutical compositions may be in the form of a salt, and may preferably further comprise a pharmaceutically acceptable carrier.

Also provided in the present invention is a method of modulating LuxS activity comprising contacting LuxS with a modulator designed or identified according to the present invention. Preferred methods include methods of treating a disease or condition associated with inappropriate LuxS activity comprising the method of administering by, for example, contacting cells of an individual with a LuxS modulator designed or identified according to the present invention. The term “inappropriate activity” refers to LuxS activity that is higher or lower than that in normal cells.

Also provided in the present invention is a method for obtaining structural information about a molecule or a molecular complex of unknown structure comprising: crystallizing the molecule or molecular complex; generating an x-ray diffraction pattern from the crystallized molecule or molecular complex; and using a molecular replacement method to interpret the structure of said molecule; wherein said molecular replacement method uses the structure coordinates of Table 7, 8, 9, 10, 11, or 12, or structure coordinates having a root mean square deviation for the alpha-carbon atoms of said structure coordinates of up to about 2.0 Å, preferably up to about 1.75 Å, preferably up to about 1.5 Å, preferably up to about 1.0 Å, preferably up to about 0.75 Å, the structure coordinates of the binding pocket of Table 7, 8, 9, 10, 11, or 12, or a binding pocket homolog.

In yet another aspect of the invention, a method is provided for homology modeling of a LuxS homolog comprising: aligning the amino acid sequence of a LuxS homolog with an amino acid sequence of LuxS; incorporating the sequence of the LuxS homolog into a model of the structure of LuxS, wherein said model has the same structure coordinates as the structure coordinates of Table 7, 8, 9, 10, 11, or 12, or wherein the structure coordinates of said model's alpha-carbon atoms have a root mean square deviation from the structure coordinates of Table 7, 8, 9, 10, 11, or 12, of up to about 2.0 Å, preferably up to about 1.75 Å, preferably up to about 1.5 Å, preferably up to about 1.0 Å, and preferably up to about 0.75 Å, to yield a preliminary model of said homolog; subjecting the preliminary model to energy minimization to yield an energy minimized model; and remodeling regions of the energy minimized model where stereochemistry restraints are violated to yield a final model of said homolog.

The invention is illustrated by way of the present application, including working examples demonstrating the crystallization LuxS, the characterization of crystals, the collection of diffraction data, and the determination and analysis of the three-dimensional structure of LuxS.

### 5. BRIEF DESCRIPTION OF THE FIGURES AND TABLES

### 5.1 Brief Description of the Figures

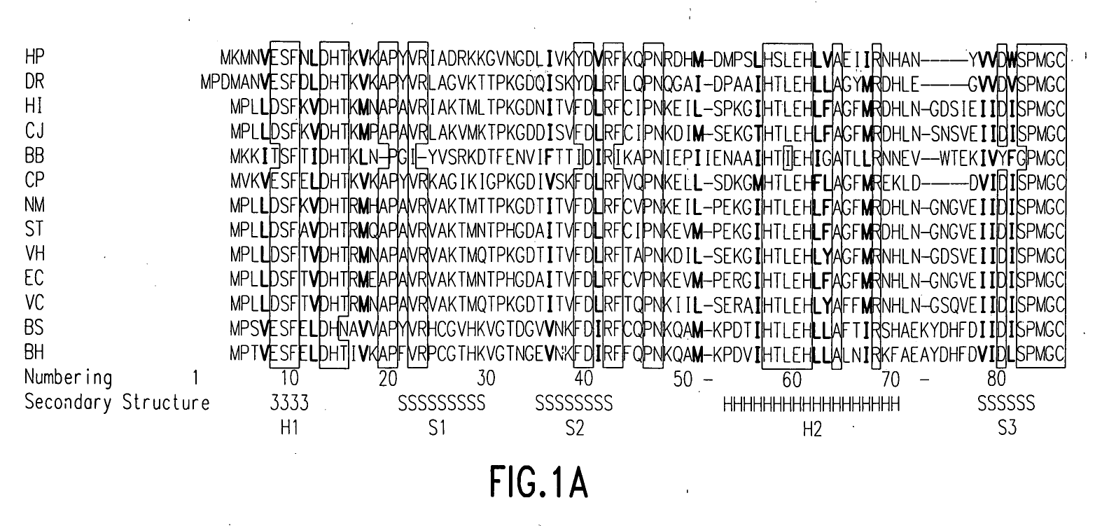

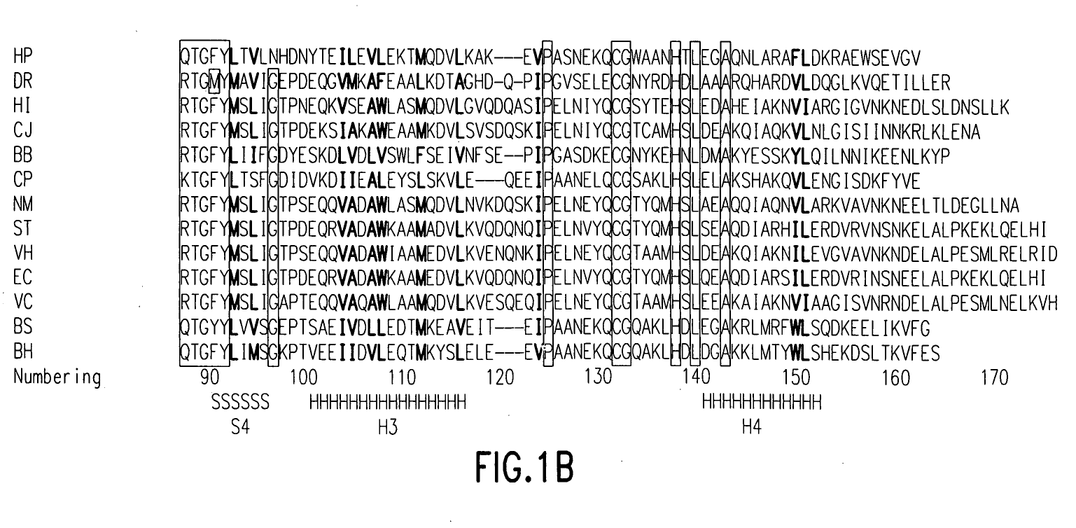

FIG. 1 provides a structure-based sequence alignment of LuxS proteins: A gray field with black characters indicates residues with greater than 92% identity (or homology in the case of F/Y, S/T, or D/E). A black field with white characters indicates hydrophobic residues (A/V/I/L/M/W/Y/F). At the bottom is indicated the residue numbering employed as well as the common secondary structure elements determined in this invention with 3=3/10 helix, S=beta strand, and H=alpha helix.

FIG. 2 provides a diffraction pattern of LuxS from H. pylori;

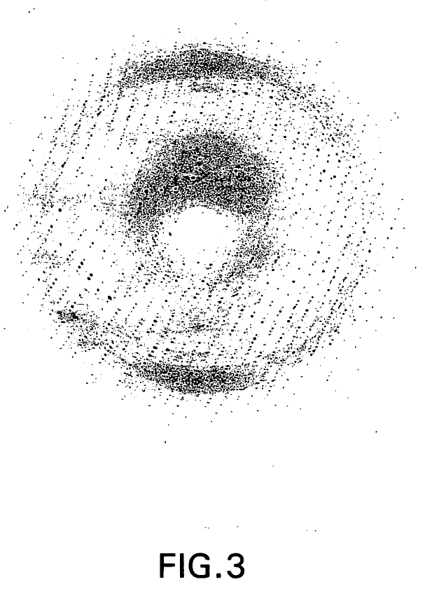

FIG. 3 provides a diffraction pattern of LuxS from H. influenzae;

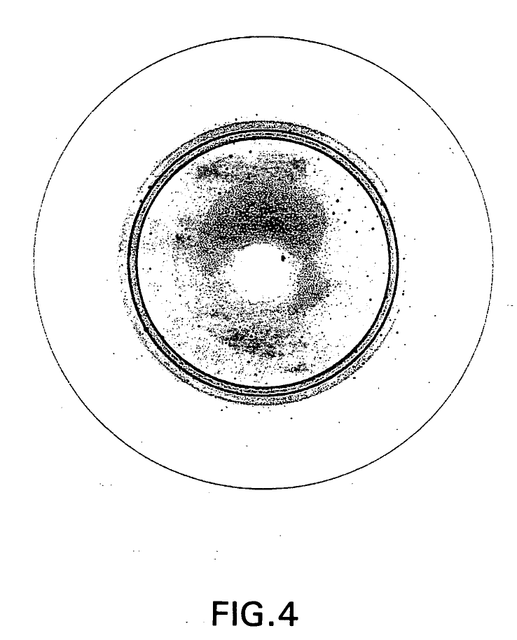

FIG. 4 provides a diffraction pattern of LuxS from D. radiodurans, P2₁ space group;

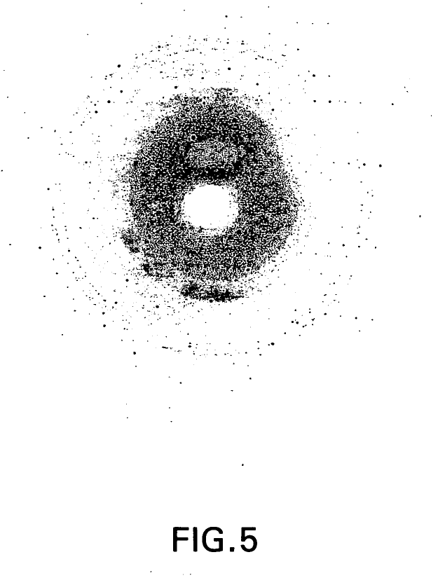

FIG. 5 provides a diffraction pattern of LuxS from D. radiodurans, C2 space group;

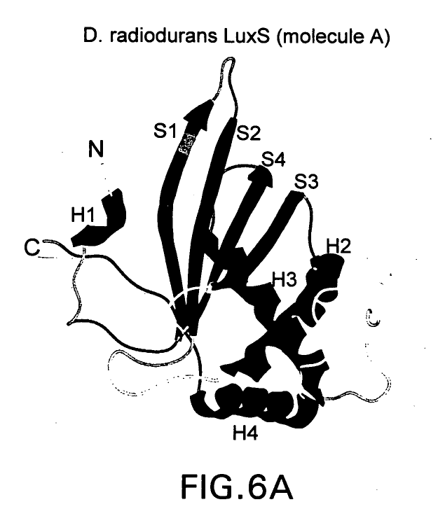

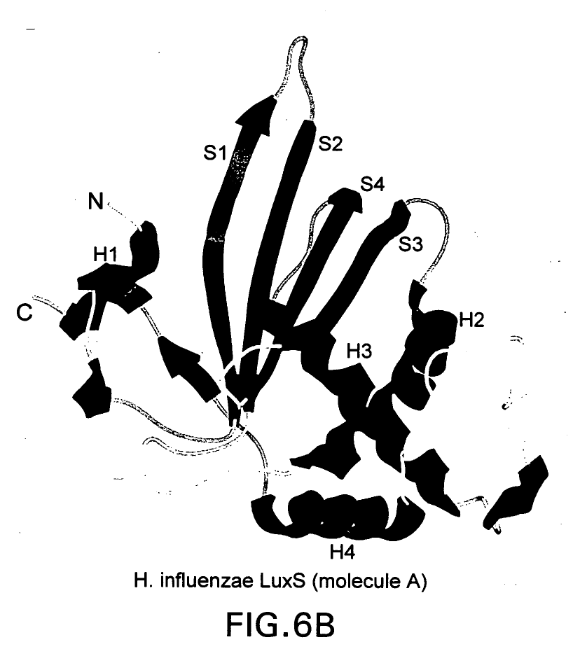

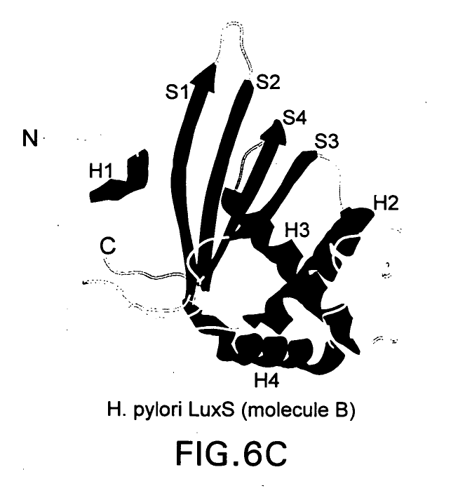

FIG. 6A provides a ribbon diagram of molecule A of the D. radiodurans LuxS structure;

FIG. 6B provides a ribbon diagram of molecule A of the H. influenzae LuxS structure;

FIG. 6C provides a ribbon diagram of molecule B of the H. pylori P2₁ LuxS structure;

FIG. 6D provides a ribbon diagram of the H. pylori C2 LuxS structure;

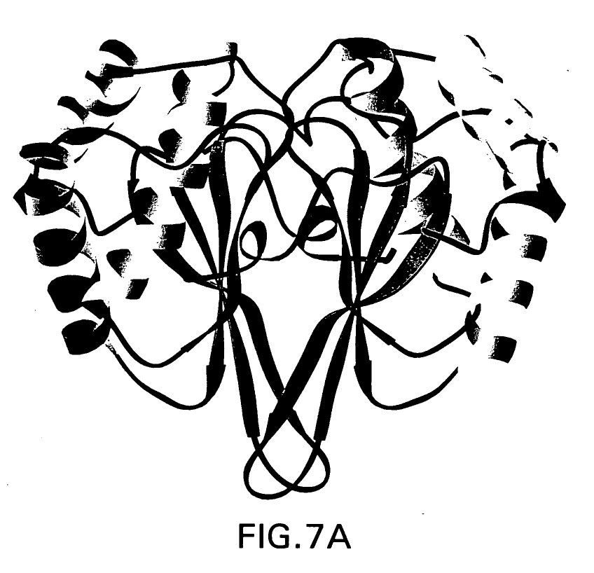

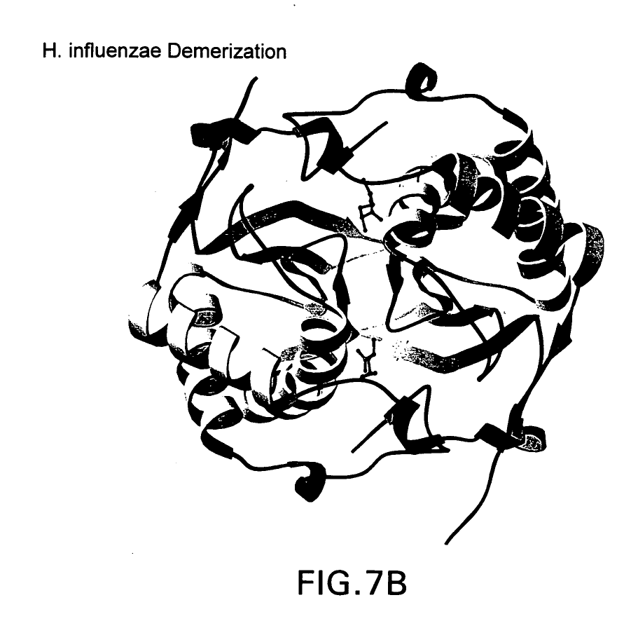

FIG. 7 provides a ribbon diagram of the H. pylori LuxS asymmetric unit contents;

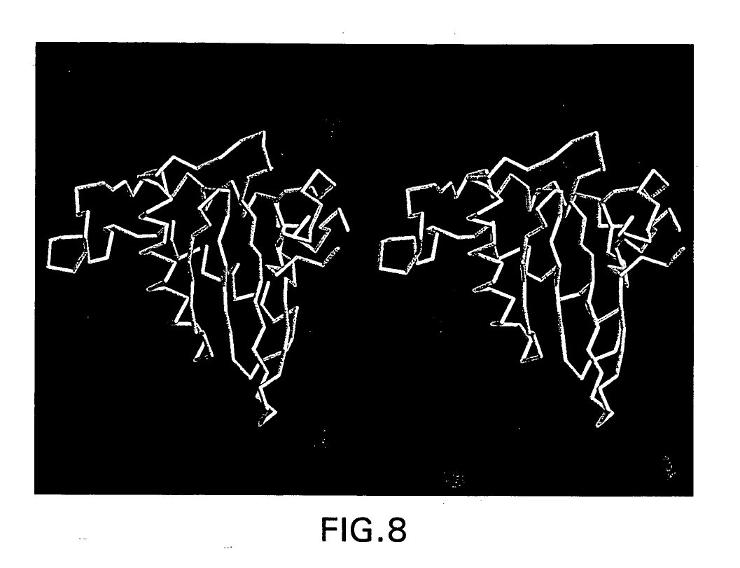

FIG. 8 provides a stereo view of a Cα trace of H. pylori LuxS.

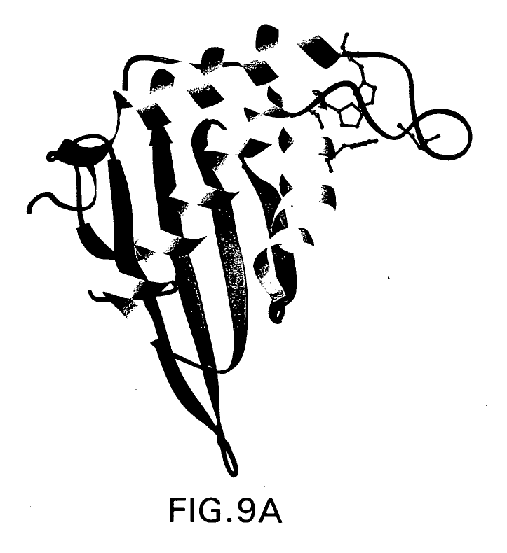

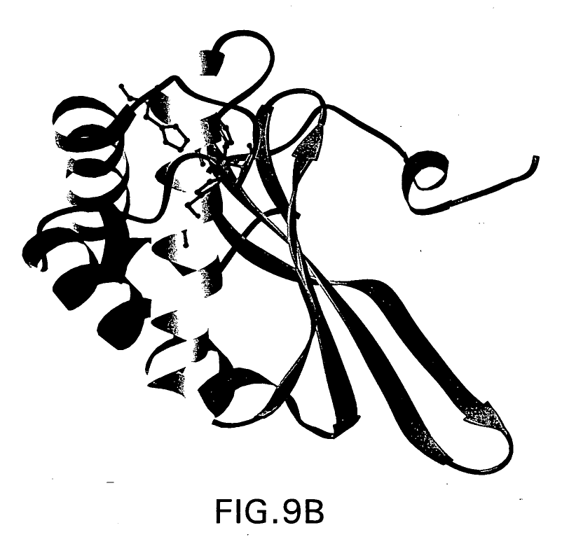

FIG. 9A provides a ribbon diagram illustrating the sequence-variable region of H. pylori LuxS;

FIG. 9B provides a ribbon diagram illustrating a sequence-conserved region of H. pylori LuxS;

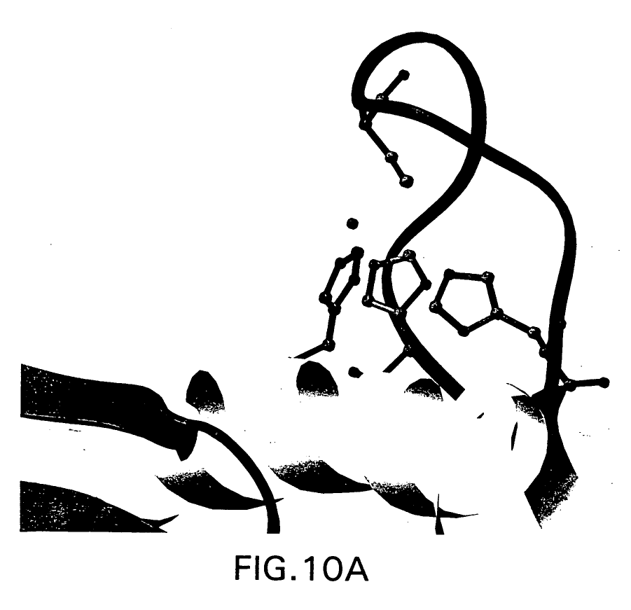

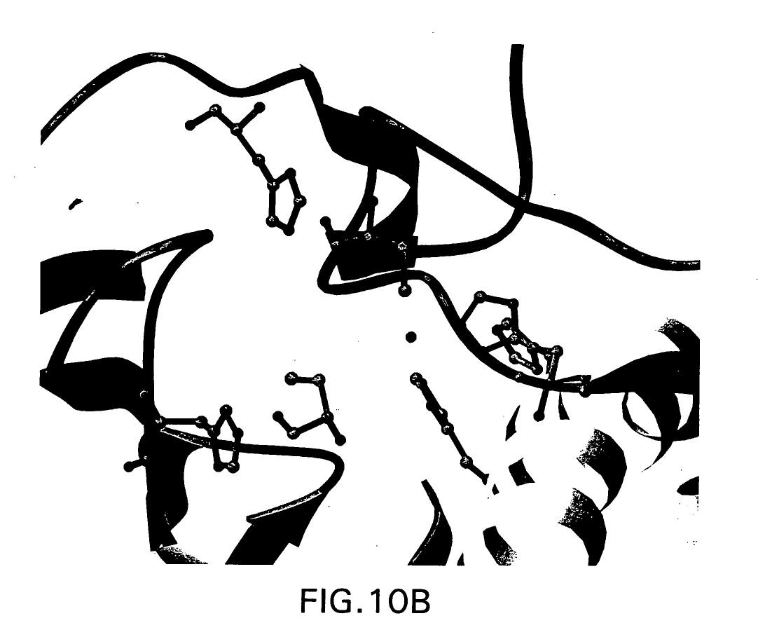

FIG. 10A provides a ribbon diagram illustrating the substrate binding site region of H. pylori LuxS as a monomer;

FIG. 10B provides a ribbon diagram illustrating the active site region of H. pylori LuxS as a dimer;

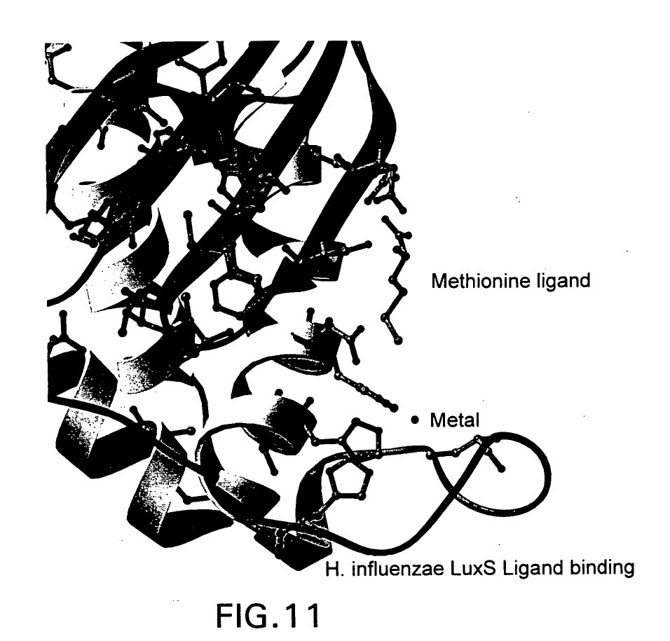

FIG. 11 provides a ribbon diagram of the active site region of H. influenzae LuxS showing the metal and methionine binding sites;

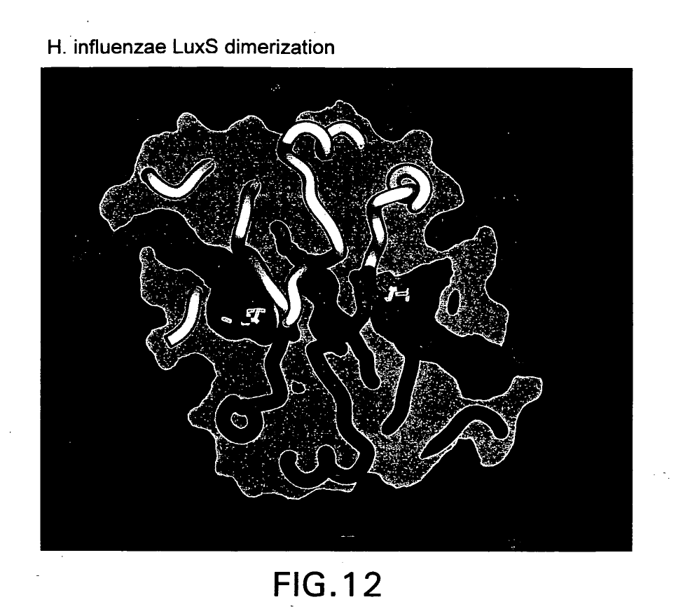

FIG. 12 provides a SPOCK diagram of the molecular surfaces of the two molecules in the asymmetric unit of H. influenzae LuxS;

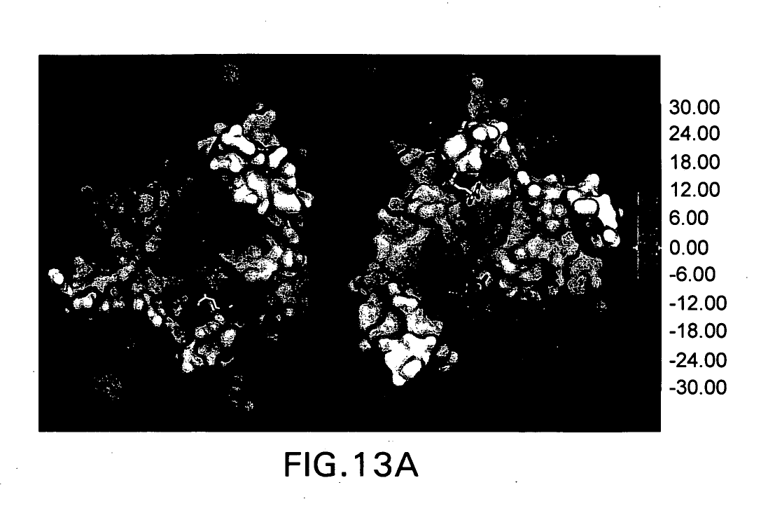

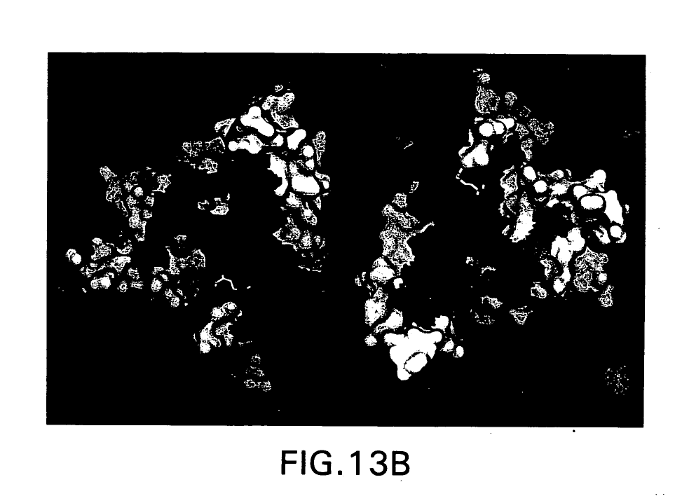

FIG. 13A illustrates the electrostatic potential of the molecular surfaces of the dimerization interface of H. influenzae LuxS; and

FIG. 13B illustrates the location of conserved residues of the dimerization interface of H. influenzae LuxS.

### 5.2 BRIEF DESCRIPTION OF THE TABLES

Table 1 shows the classifications of commonly encountered amino acids;

Table 2 summarizes the X-ray crystallography data sets of LuxS crystals that were used to determine the structures of crystalline LuxS of the inventions as well as the results of the refinements;

Table 3 presents an index of the diffraction image shown in FIG. 2;

Table 4 presents an index of the diffraction image shown in FIG. 3;

Table 5 presents an index of the diffraction image shown in FIG. 4;

Table 6 presents an index of the diffraction image shown in FIG. 5;

Table 7 summarizes the atomic structure coordinates of H. polori LuxS;

Table 8 summarizes the atomic structure coordinates of H. influenzae LuxS;

Table 9 summarizes the atomic structure coordinates of D. radiodurans P2₁ LuxS;

Table 10 summarizes the atomic structure coordinates of D. radiodurans C2 LuxS, with methionine present in the purification conditions; and

Table 11 summarizes the atomic structure coordinates of D. radiodurans C2 LuxS without added methionine in the purification conditions.

Table 12 summarizes the atomic structure coordinates of D. radiodurans P1 LuxS without added methionine in the purification conditions, and with added SeMet in the crystallization conditions.

### 6. DETAILED DESCRIPTION OF THE INVENTION

### 6.1 Abbreviations

The amino acid notations used herein for the twenty genetically encoded L-amino acids are conventional and are as follows:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when polypeptide sequences are presented as a series of one-letter and/or three-letter abbreviations, the sequences are presented in the N→C direction, in accordance with common practice.

### 6.2 Definitions

As used herein, the following terms shall have the following meanings:

“Genetically Encoded Amino Acid” refers to the twenty amino acids that are defined by genetic codons. The genetically encoded amino acids are glycine and the L-isomers of alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

“Non-Genetically Encoded Amino Acid” refers to amino acids that are not defined by genetic codons. Non-genetically encoded amino acids include derivatives or analogs of the genetically-encoded amino acids that are capable of being enzymatically incorporated into nascent polypeptides using conventional expression systems, such as selenomethionine (SeMet) and selenocysteine (SeCys); isomers of the genetically-encoded amino acids that are not capable of being enzymatically incorporated into nascent polypeptides using conventional expression systems, such as D-isomers of the genetically-encoded amino acids; L- and D-isomers of naturally occurring -amino acids that are not defined by genetic codons, such as -aminoisobutyric acid (Aib); L- and D-isomers of synthetic -amino acids that are not defined by genetic codons; and other amino acids such as -amino acids, -amino acids, etc. In addition to the D-isomers of the genetically-encoded amino acids, common genetically non-encoded amino acids include, but are not limited to norleucine (Nle), penicillamine (Pen), N-methylvaline (MeVal), homocysteine (hCys), homoserine (hSer), 2,3-diaminobutyric acid (Dab) and omithine (Orn). Additional exemplary genetically non-encoded amino acids are found, for example, in Practical Handbook of Biochemistry and Molecular Biology, 1989, Fasman, Ed., CRC Press, Inc., Boca Raton, Fla., pp. 3-76 and the various references cited therein.

“Hydrophilic Amino Acid” refers to an amino acid having a side chain exhibiting a hydrophobicity of up to about zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R). Genetically non-encoded hydrophilic amino acids include the D-isomers of the above-listed genetically-encoded amino acids, omithine (Orn), 2,3-diaminobutyric acid (Dab) and homoserine (hSer).

“Acidic Amino Acid” refers to a hydrophilic amino acid having a side chain pK value of up to about 7 under physiological conditions. Acidic amino acids typically have negatively charged side chains at physiological pH due to loss of a hydrogen ion. Genetically encoded acidic amino acids include Glu (E) and Asp (D). Genetically non-encoded acidic amino acids include D-Glu (e) and D-Asp (d).

“Basic Amino Acid” refers to a hydrophilic amino acid having a side chain pK value of greater than 7 under physiological conditions. Basic amino acids typically have positively charged side chains at physiological pH due to association with hydronium ion. Genetically encoded basic amino acids include His (H), Arg (R) and Lys (K). Genetically non-encoded basic amino acids include the D-isomers of the above-listed genetically-encoded amino acids, omithine (Orn) and 2,3-diaminobutyric acid (Dab).

“Polar Amino Acid” refers to a hydrophilic amino acid having a side chain that is uncharged at physiological pH, but which comprises at least one covalent bond in which the pair of electrons shared in common by two atoms is held more closely by one of the atoms. Genetically encoded polar amino acids include Asn (N), Gln (Q), Ser (S), and Thr (T). Genetically non-encoded polar amino acids include the D-isomers of the above-listed genetically-encoded amino acids and homoserine (hSer).

“Hydrophobic Amino Acid” refers to an amino acid having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include Pro (P), Ile (I), Phe (F), Val (V), Leu (L), Trp (W), Met (M), Ala (A), Gly (G) and Tyr (Y). Genetically non-encoded hydrophobic amino acids include the D-isomers of the above-listed genetically-encoded amino acids, norleucine (Nle) and N-methyl valine (MeVal).

“Aromatic Amino Acid” refers to a hydrophobic amino acid having a side chain comprising at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, (C₁-C₆) alkenyl, or (C₁-C₆) alkynyl. Genetically encoded aromatic amino acids include Phe (F), Tyr (Y), Trp (W) and His (H). Genetically non-encoded aromatic amino acids include the D-isomers of the above-listed genetically-encoded amino acids.

“Apolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A). Genetically non-encoded apolar amino acids include the D-isomers of the above-listed genetically-encoded amino acids, norleucine (Nle) and N-methyl valine (MeVal).

“Aliphatic Amino Acid” refers to a hydrophobic amino acid having an aliphatic hydrocarbon side chain. Genetically encoded aliphatic amino acids include Ala (A), Val (V), Leu (L) and Ile (I). Genetically non-encoded aliphatic amino acids include the D-isomers of the above-listed genetically-encoded amino acids, norleucine (Nle) and N-methyl valine (MeVal).

“Helix-Breaking Amino Acid” refers to those amino acids that have a propensity to disrupt the structure of -helices when contained at internal positions within the helix. Amino acid residues exhibiting helix-breaking properties are well-known in the art (see, e.g., Chou & Fasman, 1978, Ann. Rev. Biochem. 47:251-276) and include Pro (P), D-Pro (p), Gly (G) and potentially all D-amino acids (when contained in an L-polypeptide; conversely, L-amino acids disrupt helical structure when contained in a D-polypeptide).

“Cysteine-like Amino Acid” refers to an amino acid having a side chain capable of participating in a disulfide linkage. Thus, cysteine-like amino acids generally have a side chain containing at least one thiol (—SH) group. Cysteine-like amino acids are unusual in that they can form disulfide bridges with other cysteine-like amino acids. The ability of Cys (C) residues and other cysteine-like amino acids to exist in a polypeptide in either the reduced free —SH or oxidized disulfide-bridged form affects whether they contribute net hydrophobic or hydrophilic character to a polypeptide. Thus, while Cys (C) exhibits a hydrophobicity of 0.29 according to the consensus scale of Eisenberg (Eisenberg, 1984, supra), it is to be understood that for purposes of the present invention Cys (C) is categorized as a polar hydrophilic amino acid, notwithstanding the general classifications defined above. Other cysteine-like amino acids are similarly categorized as polar hydrophilic amino acids. Typical cysteine-like residues include, for example, penicillamine (Pen), homocysteine (hCys), etc.

As will be appreciated by those of skill in the art, the above-defined classes or categories are not mutually exclusive. Thus, amino acids having side chains exhibiting two or more physico-chemical properties can be included in multiple categories. For example, amino acid side chains having aromatic groups that are further substituted with polar substituents, such as Tyr (Y), may exhibit both aromatic hydrophobic properties and polar or hydrophilic properties, and could therefore be included in both the aromatic and polar categories. Typically, amino acids will be categorized in the class or classes that most closely define their net physico-chemical properties. The appropriate categorization of any amino acid will be apparent to those of skill in the art.

The classifications of the genetically encoded and common non-encoded amino acids according to the categories defined above are summarized in Table 1, below. It is to be understood that Table 1 is for illustrative purposes only and does not purport to be an exhaustive list of the amino acid residues belonging to each class. Other amino acid residues not specifically mentioned herein can be readily categorized based on their observed physical and chemical properties in light of the definitions provided herein.

TABLE 1
CLASSIFICATIONS OF COMMONLY ENCOUNTERED
AMINO ACIDS

| | Genetically | Genetically |
|---|---|---|
| Classification | Encoded | Non-Encoded |
| Hydrophobic | | |
| Aromatic | F, Y, W, H | f, y, w, h |
| Apolar | L, V, I, M, G, A, P | l, v, i, m, a, p, Nle, MeVal |
| Aliphatic | A, V, L, I | a, v, l, i, Nle, MeVal |
| Hydrophilic | | |
| Acidic | D, E | d, e |
| Basic | H, K, R | h, k, r, Orn, Dab |
| Polar | C, Q, N, S, T | c, q, n, s, t, hSer |
| Helix-Breaking | P, G | p |

“LuxS polypeptide” or “LuxS” refers to a polypeptide comprising an amino acid sequence that corresponds to a wild-type LuxS polypeptide or a mutant LuxS polypeptide, as defined below.

“Wild-type LuxS polypeptide” or “wtLuxS” refers to a polypeptide comprising an amino acid sequence that corresponds identically to the amino acid sequence of a naturally-occurring LuxS.

“Helicobacter pylori wtLuxS” refers to a polypeptide comprising an amino acid sequence that corresponds identically to the wild-type LuxS from Helicobacter pylori (FIG. 1, SEQ ID NO:1).

“Haemophilus influenzae wtLuxS” refers to a polypeptide comprising an amino acid sequence that corresponds identically to the wild-type LuxS from Haemophilus influenzae (FIG. 1, SEQ ID NO:2).

“Deinococcus radiodurans wtLuxS” refers to a polypeptide comprising an amino acid sequence that corresponds identically to the wild-type LuxS from Deinococcus radiodurans (FIG. 1, SEQ ID NO:3).

“Crystallized Helicobacter pylori LuxS” refers to a polypeptide comprising an amino acid sequence which corresponds identically to SEQ ID NO: 1, or a mutant thereof, and which is in crystalline form.

“Crystallized Haemophilus influenzae LuxS” refers to a polypeptide comprising an amino acid sequence which corresponds identically to SEQ ID NO: 2, or a mutant thereof, and which is in crystalline form.

“Crystallized Deinococcus radiodurans LuxS” refers to a polypeptide comprising an amino acid sequence which corresponds identically to SEQ ID NO: 3, or a mutant thereof, and which is in crystalline form.

“Association” refers to a condition of proximity between a chemical entity or compound, or portions or fragments thereof, and a polypeptide, or portions or fragments thereof. The association may be non-covalent, i.e., where the juxtaposition is energetically favored by, e.g., hydrogen-bonding, van der Waals, electrostatic or hydrophobic interactions, or it may be covalent.

“Co-Complex” refers to a LuxS polypeptide in association with one or more compounds. Such compounds include, by way of example and not limitation, cofactors, ligands, substrates, substrate analogues, inhibitors, allosteric effectors, etc.

“Co-Complex” refers to a polypeptide in association with one or more compounds. Such compounds include, by way of example and not limitation, cofactors, ligands, substrates, substrate analogues, inhibitors, allosteric effectors, etc. Preferred lead compounds for designing LuxS inhibitors include, but are not restricted to, S-ribosyl-homocysteine and methionine. A co-complex may also refer to a computer represented, or in silica generated association between a peptide and a compound. An “unliganded” form of a protein structure, or structural coordinates thereof, refers to the coordinates of a peptide that is not part of a co-complex. A “liganded” form refers to the coordinates of a peptide that is part of a co-complex. Unliganded forms include peptides associated with various ions, such as manganese, zinc, and magnesium, as well as with water. Liganded forms include peptides associated with natural substrates, non-natural substrates, and small molecules, as well as, optionally, in addition, various ions or water.

“Mutant LuxS polypeptide” or “mLuxS” or “Mutant” refers to a polypeptide characterized by an amino acid sequence that differs from the wild-type sequence by the substitution of at least one amino acid residue of the wild-type sequence with a different amino acid residue and/or by the addition and/or deletion of one or more amino acid residues to or from the wild-type sequence. The additions and/or deletions can be from an internal region of the wild-type sequence and/or at either or both of the N- or C-termini. A mutant polypeptide may preferably have substantially the same three-dimensional structure as the corresponding wild-type polypeptide. A mutant may have, but need not have, LuxS activity. Preferably, a mutant displays biological activity that is substantially similar to that of the wild-type LuxS. By “substantially similar biological activity” is meant that the mutant displays biological activity that is within 1% to 10,000% of the biological activity of the wild type polypeptide, more preferably within 25% to 5,000%, and most preferably, within 50% to 500%, or 75% to 200% of the biological activity of the wild type polypeptide, using assays known to those of ordinary skill in the art for that particular class of polypeptides. Mutants may be synthesized according to any method known to those skilled in the art, including, but not limited to, those methods of expressing LuxS molecules described herein.

“Active Site” refers to a site in LuxS that associates with the substrate for LuxS activity. Preferably, the active site comprises Glu60, Arg68, Ile81, and Asp80, preferably the active site further comprises Ala64, His61, Tyr91, Ser9, Phe10, and Leu7. Preferably the active site further comprises His14, Arg23, Asp40, Arg42, Met84, Cys86, and Thr88. according to the sequence of FIG. 2. Amino acid residue numbers presented herein refer to the sequence of FIG. 2.

“Accessory Binding Site” refers to a binding site in LuxS other than that of the “active site.” One metal binding accessory binding site comprises residues His57, His61, Cys131, and His137.

“Conservative Mutant” refers to a mutant in which at least one amino acid residue from the wild-type sequence is substituted with a different amino acid residue that has similar physical and chemical properties, i.e., an amino acid residue that is a member of the same class or category, as defined above. For example, a conservative mutant may be a polypeptide that differs in amino acid sequence from the wild-type sequence by the substitution of a specific aromatic Phe (F) residue with an aromatic Tyr (Y) or Trp (W) residue.

“Non-Conservative Mutant” refers to a mutant in which at least one amino acid residue from the wild-type sequence is substituted with a different amino acid residue that has dissimilar physical and/or chemical properties, i.e., an amino acid residue that is a member of a different class or category, as defined above. For example, a non-conservative mutant may be a polypeptide that differs in amino acid sequence from the wild-type sequence by the substitution of an acidic Glu (E) residue with a basic Arg (R), Lys (K) or Orn residue.

“Deletion Mutant” refers to a mutant having an amino acid sequence that differs from the wild-type sequence by the deletion of one or more amino acid residues from the wild-type sequence. The residues may be deleted from internal regions of the wild-type sequence and/or from one or both termini.

“Truncated Mutant” refers to a deletion mutant in which the deleted residues are from the N- and/or C-terminus of the wild-type sequence.

“Extended Mutant” refers to a mutant in which additional residues are added to the N- and/or C-terminus of the wild-type sequence.

“Methionine mutant” refers to (1) a mutant in which at least one methionine residue of the wild-type sequence is replaced with another residue, preferably with an aliphatic residue, most preferably with an Ala (A), Leu (L), or Ile (I) residue; or (2) a mutant in which a non-methionine residue, preferably an aliphatic residue, most preferably an Ala (A), Leu (L) or Ile (I) residue, of the wild-type sequence is replaced with a methoinine residue.

“Selenomethionine mutant” refers to (1) a mutant which includes at least one selenomethionine (SeMet) residue, typically by substitution of a Met residue of the wild-type sequence with a SeMet residue, or by addition of one or more SeMet residues at one or both termini, or (2) a methionine mutant in which at least one Met residue is substituted with a SeMet residue. Preferred SeMet mutants are those in which each Met residue is substituted with a SeMet residue.

“Cysteine mutant” refers to (1) a mutant in which at least one cysteine residue of the wild-type sequence is replaced with another residue, preferably with a Ser (S) residue; or (2) a mutant in which a non-cysteine residue, preferably a Ser (S) residue, of the wild-type sequence is replaced with a cysteine residue.

“Selenocysteine mutant” refers to (1) a mutant which includes at least one selenocysteine (SeCys) residue, typically by substitution of a Cys residue of the wild-type sequence with a SeCys residue, or by addition of one or more SeCys residues at one or both termini, or (2) a cysteine mutant in which at least one Cys residue is substituted with a SeCys residue. Preferred SeCys mutants are those in which each Cys residue is substituted with a SeCys residue.

“Homolog” refers to a polypeptide having at least 30%, preferably at least 40%, preferably at least 50%, preferably at least 60%, preferably at least 70%, more preferably at least 80%, and most preferably at least 90% amino acid sequence identity or having a BLAST score of 1×10⁻⁶ over at least 100 amino acids (Altschul et al., 1997, Nucleic Acids Res. 25:3389-402) with LuxS or any functional domain of LuxS.

“Crystal” refers to a composition comprising a polypeptide in crystalline form. The term “crystal” includes native crystals, heavy-atom derivative crystals and co-crystals, as defined herein.

“Native Crystal” refers to a crystal wherein the polypeptide is substantially pure. As used herein, native crystals do not include crystals of polypeptides comprising amino acids that are modified with heavy atoms, such as crystals of selenomethionine mutants, selenocysteine mutants, etc.

“Heavy-atom Derivative Crystal” refers to a crystal wherein the polypeptide is in association with one or more heavy-metal atoms. As used herein, heavy-atom derivative crystals include native crystals into which a heavy metal atom is soaked, as well as crystals of selenomethionine mutants and selenocysteine mutants.

“Co-Crystal” refers to a composition comprising a co-complex, as defined above, in crystalline form. Co-crystals include native co-crystals and heavy-atom derivative co-crystals.

“Apo-crystal” refers to a crystal wherein the polypeptide is substantially pure and substantially free of compounds that might form a co-complex with the polypeptide such as cofactors, ligands, substrates, substrate analogues, inhibitors, allosteric effectors, etc.

“Diffraction Quality Crystal” refers to a crystal that is well-ordered and of a sufficient size, i.e., at least 10 μm, preferably at least 50 μm, and most preferably at least 100 μm in its smallest dimension such that it produces measurable diffraction to at least 3 Å resolution, preferably to at least 2 Å resolution, and most preferably to at least 1.5 Å resolution or lower. Diffraction quality crystals include native crystals, heavy-atom derivative crystals, and co-crystals.

“Unit Cell” refers to the smallest and simplest volume element (i.e., parallelepiped-shaped block) of a crystal that is completely representative of the unit or pattern of the crystal, such that the entire crystal can be generated by translation of the unit cell. The dimensions of the unit cell are defined by six numbers: dimensions a, b and c and the angles are defined as α, β, and γ. (Blundell et al., 1976, Protein Crystallography, 83-84, Academic Press.). A crystal is an efficiently packed array of many unit cells.

“Triclinic Unit Cell” refers to a unit cell in which a≠b≠c and α≠β≠γ.

“Monoclinic Unit Cell” refers to a unit cell in which a≠b≠c; and α=γ=90°; and β>90°.

“Hexagonal Unit Cell” refers to a unit cell in which a=b≠c; α=β=90°; and γ=120°.

“Orthorhombic Unit Cell” refers to a unit cell in which a≠b≠c; and α=β=γ=90°.

“Tetragonal Unit Cell” refers to a unit cell in which a=b≠c; and α=β=γ=90°.

“Trigonal/Rhombohedral Unit Cell” refers to a unit cell in which a=b=c; and α=β=γ≠90°.

“Trigonal/Hexagonal Unit Cell” refers to a unit cell in which a=b≠c; α=β=90°; and γ=120°.

“Cubic Unit Cell” refers to a unit cell in which a=b=c; and α=β=γ=90°.

“Crystal Lattice” refers to the array of points defined by the vertices of packed unit cells.

“Space Group” refers to the set of symmetry operations of a unit cell. In a space group designation (e.g., C2) the capital letter indicates the lattice type and the other symbols represent symmetry operations that can be carried out on the unit cell without changing its appearance.

“Asymmetric Unit” refers to the largest aggregate of molecules in the unit cell that possesses no symmetry elements that are part of the space group symmetry, but that can be juxtaposed on other identical entities by symmetry operations.

“Crystallographically-Related Dimer (or oligomer)” refers to a dimer (or oligomer, such as, for example, a trimer or a tetramer) of two (or more) molecules wherein the symmetry axes or planes that relate the two (or more) molecules comprising the dimer (or oligomer) coincide with the symmetry axes or planes of the crystal lattice.

“Non-Crystallographically-Related Dimer (or oligomer)” refers to a dimer (or oligomer, such as, for example, a trimer or a tetramer) of two (or more) molecules wherein the symmetry axes or planes that relate the two (or more) molecules comprising the dimer (or oligomer) do not coincide with the symmetry axes or planes of the crystal lattice.

“Isomorphous Replacement” refers to the method of using heavy-atom derivative crystals to obtain the phase information necessary to elucidate the three-dimensional structure of a crystallized polypeptide (Blundell et al., 1976, Protein Crystallography, Academic Press, esp. pp. 151-164; Methods in Enzymology 276:361-557 Academic Press, 1997). The phrase “heavy-atom derivatization” is synonymous with “isomorphous replacement.”

“Multi-Wavelength Anomalous Dispersion or MAD” refers to a crystallographic technique in which X-ray diffraction data are collected at several different wavelengths from a single heavy-atom derivative crystal, wherein the heavy atom has absorption edges near the energy of incoming X-ray radiation. The resonance between X-rays and electron orbitals leads to differences in X-ray scattering from absorption of the X-rays (known as anomalous scattering) and permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, Science, 254:51-58, 1991.

“Single Wavelength Anomalous Dispersion or SAD” refers to a crystallographic technique in which X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique needs to be close to the absorption edge of the anomalous scatterer. A detailed discussion of SAD analysis can be found in Brodersen et al., 2000, Acta Cryst., D56:431-441.

“Single Isomorphous Replacement With Anomalous Scattering or SIRAS” refers to a crystallographic technique that combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

“Molecular Replacement” refers to the method using the structure coordinates of a known polypeptide to calculate initial phases for a new crystal of a polypeptide whose structure coordinates are unknown. This is done by orienting and positioning a polypeptide whose structure coordinates are known within the unit cell of the new crystal. Phases are then calculated from the oriented and positioned polypeptide and combined with observed amplitudes to provide an approximate Fourier synthesis of the structure of the polypeptides comprising the new crystal. The model is then refined to provide a refined set of structure coordinates for the new crystal. (Lattman, 1985, Methods in Enzymology, 115:55-77; Rossmann, 1972, “The Molecular Replacement Method,” Int. Sci. Rev. Ser. No. 13, Gordon & Breach, New York; Methods in Enzymology, Vols. 276, 277 (Academic Press, San Diego 1997)). Molecular replacement may be used, for example, to determine the structure coordinates of a crystalline mutant or homolog of LuxS using the structure coordinates of LuxS.

“Structure coordinates” refers to mathematical coordinates derived from mathematical equations related to the patterns obtained on diffraction of a monochromatic beam of X-rays by the atoms (scattering centers) of a LuxS in crystal form. The diffraction data are used to calculate an electron density map of the repeating unit of the crystal. The electron density maps are used to establish the positions of the individual atoms within the unit cell of the crystal.

“Having substantially the same three-dimensional structure” refers to a polypeptide that is characterized by a set of molecular structure coordinates that have a root mean square deviation (r.m.s.d.) of up to about or equal to 2 Å, preferably 1.75 Å, preferably 1.5 Å, and preferably 1.0 Å, and preferably 0.75 Å, when superimposed onto the molecular structure coordinates of FIG. 4 when at least 50% to 100% of the C-alpha atoms of the coordinates are included in the superposition. The program MOE may be used to compare two structures. Where structure coordinates are not available for a particular amino acid residue(s), those coordinates are not included in the calculation.

“α-C” or “α-carbon” As used herein, “α-C” or “α-carbon” refer to the alpha carbon of an amino acid residue.

“α-helix” refers to the conformation of a polypeptide chain in the form of a spiral chain of amino acids stabilized by hydrogen bonds.

The term “β-sheet” refers to the conformation of a polypeptide chain stretched into an extended zig-zig conformation. Portions of polypeptide chains that run “parallel” all run in the same direction. Where polypeptide chains are “antiparallel,” neighboring chains run in opposite directions from each other. The term “run” refers to the N to COOH direction of the polypeptide chain.

6.3 Crystalline LuxS

The crystals from which the atomic structure coordinates of the invention may be obtained include native crystals and heavy-atom derivative crystals. Native crystals generally comprise substantially pure polypeptides corresponding to LuxS in crystalline form.

It is to be understood that the crystalline LuxS from which the atomic structure coordinates of the invention can be obtained is not limited to wild-type LuxS. Indeed, the crystals may comprise mutants of wild-type LuxS. Mutants of wild-type LuxS are obtained by replacing at least one amino acid residue in the sequence of the wild-type LuxS with a different amino acid residue, or by adding or deleting one or more amino acid residues within the wild-type sequence and/or at the N- and/or C-terminus of the wild-type LuxS. Preferably, such mutants will crystallize under crystallization conditions that are substantially similar to those used to crystallize the wild-type LuxS.

The types of mutants contemplated by this invention include conservative mutants, non-conservative mutants, deletion mutants, truncated mutants, extended mutants, methionine mutants, selenomethionine mutants, cysteine mutants and elenocysteine mutants. A mutant may have, but need not have, LuxS activity. Preferably, a mutant displays biological activity that is substantially similar to a biological activity of the wild-type polypeptide. Methionine, selenomethione, cysteine, and selenocysteine mutants are particularly useful mutants, as they may be used to produce heavy-atom derivative crystals, as described in detail, below.

It will be recognized by one of skill in the art that the types of mutants contemplated herein are not mutually exclusive; that is, for example, a polypeptide having a conservative mutation in one amino acid may in addition have a truncation of residues at the N-terminus, and several Leu or Ile→Met mutations.

Sequence alignments of polypeptides in a LuxS family or of homologous polypeptide domains can be used to identify potential amino acid residues in the polypeptide sequence that are candidates for mutation (see FIG. 1). Identifying mutations that do not significantly interfere with the three-dimensional structure of LuxS and/or that do not deleteriously affect, and that may even enhance, a biological activity of LuxS will depend, in part, on the region where the mutation occurs. In highly variable regions of the molecule, such as those shown in FIG. 9A, non-conservative substitutions as well as conservative substitutions may be tolerated without significantly disrupting the three-dimensional structure and/or biological activity of the molecule. In highly conserved regions, or regions containing significant secondary structure, such as those regions shown in FIG. 9B, conservative amino acid substitutions are preferred.

Conservative amino acid substitutions are well-known in the art, and include substitutions made on the basis of a similarity in polarity, charge, solubility, hydrophobicity and/or the hydrophilicity of the amino acid residues involved. Typical conservative substitutions are those in which a wild type amino acid is substituted with a different amino acid that is a member of the same class or category, as those classes are defined herein. Thus, typical conservative substitutions include aromatic to aromatic, apolar to apolar, aliphatic to aliphatic, acidic to acidic, basic to basic, polar to polar, etc. Other conservative amino acid substitutions are well known in the art. It will be recognized by those of skill in the art that generally, a total of about 20% or fewer, typically about 10% or fewer, most usually about 5% or fewer, of the amino acids in the wild-type polypeptide sequence can be conservatively substituted with other amino acids without deleteriously affecting the biological activity and/or three-dimensional structure of the molecule, provided that such substitutions do not involve residues that are critical for activity, as discussed above.

Inspection of the various LuxS structures of the invention reveals that a metal binding site is a likely candidate for being at least in part responsible for the known enzymatic activity of the wtLuxS polypeptide. The residues involved in the metal binding are His 57, His 61, Cys 131, and His 137 (“metal binding site”). EXAFS data on LuxS crystals clearly indicate this metal is a zinc atom or ion. Nearby, on the same face of the LuxS polypepetide, is bound a methionine molecule. This methionine is specifically held by its backbone atoms through hydrogen bonds with residues Arg 68, Asp 80, and Ile 81 (“amino acid binding site”). The sidechain of the bound methionine points toward the metal binding site, an arrangement that is common for substrates of metalloenzymes thereby indicating that the bound methionine might be bound at a substrate binding site of LuxS (see FIG. 11). The metal and amino acid binding sites both lay at the interface of the homodimer, indicating the biological relevance of the homodimer in the function of LuxS.

Methionine is not a likely substrate of LuxS. However, modeling of a potential substrate of LuxS into the site occupied by the bound methionine residue indicated a potential active site of the enzyme. The potential substrate, S-ribosylhomocysteine contacted several residues of the LuxS polypeptide including Ser 9, His 14, Arg 23, Asp 40, Arg 42, Glu 60, Met 84, Cyc 86, Thr 88, and Tyr 91 (“active site”). The active site of LuxS is described in detail below. In addition, all four LuxS polypeptides crystallized as dimers. The structures of LuxS revealed that several residues contribute to the dimerization interface including His 14, Pro 46, Asn 47, residue 50 (usually a hydrophobic residue), His 57, Glu 60, Cys 86, residue 87 (usually Arg), residue 128 (usually Glu), residue 129 (usually hydrophobic), Gly 132, residue 134 (usually hydrophobic) and residue (usually hydrophobic). These residues comprise the dimerization interface of LuxS (“dimerization interface”). Those of skill in the art will recognize that each region identified in a LuxS polypeptide can also be identified in other LuxS polypeptides with one or more conservative or non-conservative mutations at any residue of the region.

In some embodiments, it may be desirable to make mutations in the metal binding site, the amino acid binding site, the active site or the dimerization interface of a LuxS, e.g., to reduce or completely eliminate LuxS activity. For example, it may be desirable to mutate important residues in one or more of these sites of LuxS including, for example, His 57, His 61, Cys 131, His 137, Arg 68, Asp 80 or Ile 81. Other mutations that will reduce or completely eliminate the activity of a particular LuxS will be apparent to those of skill in the art.

While in most instances the amino acids of wtLuxS will be substituted with genetically-encoded amino acids, in certain circumstances mutants may include genetically non-encoded amino acids. For example, non-encoded derivatives of certain encoded amino acids, such as SeMet and/or SeCys, may be incorporated into the polypeptide chain using biological expression systems (such SeMet and SeCys mutants are described in more detail, infra).

Alternatively, in instances where the mutant will be prepared in whole or in part by chemical synthesis, virtually any non-encoded amino acids may be used, ranging from D-isomers of the genetically encoded amino acids to non-encoded naturally-occurring natural and synthetic amino acids.

Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

In some instances, it may be particularly advantageous or convenient to substitute, delete from and/or add residues to wtLuxS in order to provide convenient cloning sites in cDNA encoding the polypeptide, to aid in purification of the polypeptide, etc. Such substitutions, deletions and/or additions that do not substantially alter the three dimensional structure of the wtLuxS will be apparent to those having skills in the art. These substitutions, deletions and/or additions include, but are not limited to, His tags, intein-containing self-cleaving tags, fusions of wtLuxS with other peptides, proteins, polypeptides or proteins such as maltose binding protein, glutathione S-transferase, antibodies, green fluorescent proteins, signal peptides, biotin accepting peptides, and the like. For instance, the LuxS polypeptides whose structures were determined as described in the Examples below possessed C-terminal His-tags (i.e. a Gly-Ser-His-His-His-His-His-His sequence added recombinantly to the C-terminal ends of the sequences in FIG. 1).

Mutations may also be introduced into a polypeptide sequence where there are residues, e.g., cysteine residues, that interfere with crystallization. Such cysteine residues can be substituted with an appropriate amino acid that does not readily form covalent bonds with other amino acid residues under crystallization conditions; e.g., by substituting the cysteine with Ala, Ser or Gly. Any cysteine located in a non-helical or non-β-stranded segment, based on secondary structure assignments, are good candidates for replacement. There are only two cysteine residues in the LuxS polypeptide crystallized in this invention and one, and possibly both are involved in the putative active site of this enzyme. Mutation of one or both of these residues would likely not effect the fold (structure) of this polypeptide but could likely effect an activity of the protein. Conservative mutation of such cysteine residues are preferred where active LuxS polypeptides are desired. Non-conservative mutation of such cysteine residues are preferable when a biological activity of LuxS, such as metal ion binding, can be reduced or eliminated. Such non-conservative mutants can be used to, for example, study the allosteric effects of metal ion binding on LuxS.

It should be noted that the mutants contemplated herein need not exhibit LuxS activity. Indeed, amino acid substitutions, additions or deletions that interfere with the activity of LuxS are specifically contemplated by the invention. Such crystalline polypeptides, or the atomic structure coordinates obtained therefrom, can be used to provide phase information to aid the determination of the three-dimensional X-ray structures of other related or non-related crystalline polypeptides.

The heavy-atom derivative crystals from which the atomic structure coordinates of the invention are obtained generally comprise a crystalline LuxS polypeptide in association with one or more heavy metal atoms. The polypeptide may correspond to a wild-type or a mutant LuxS, which may optionally be in co-complex with one or more molecules, as previously described. There are two types of heavy-atom derivatives of polypeptides. In one form, heavy-atom derivatives result from exposure of the LuxS to a heavy metal in solution, wherein crystals are grown in medium comprising the heavy metal, or in crystalline form, wherein the heavy metal diffuses into the crystal. In another form, heavy-atom derivatives comprise polypeptides having heavy-atom containing amino acids, e.g., selenomethionine and/or selenocysteine mutants.

In practice, heavy-atom derivatives of the first type can be formed by soaking a native crystal in a solution comprising heavy metal atom salts, or organometallic compounds, e.g., lead chloride, gold thiomalate, ethylmercurithiosalicylic acid-sodium salt (thimerosal), uranyl acetate, platinum tetrachloride, osmium tetraoxide, zinc sulfate, and cobalt hexamine, which can diffuse through the crystal and bind to the crystalline polypeptide.

Heavy-atom derivatives of this type can also be formed by adding to a crystallization solution comprising the polypeptide to be crystallized an amount of a heavy metal atom salt, which may associate with the LuxS and be incorporated into the crystal. The location(s) of the bound heavy metal atom(s) can be determined by X-ray diffraction analysis of the crystal. This information, in turn, is used to generate the phase information needed to construct the three-dimensional structure of the crystalline LuxS.

Heavy-atom derivative crystals may also be prepared from polypeptides that include one or more SeMet and/or SeCys residues (SeMet and/or SeCys mutants). Such selenocysteine or selenomethionine mutants may be made from wild-type or mutant LuxS by expression of LuxS-encoding cDNAs in auxotrophic E. coli strains (Hendrickson et al., 1990, EMBO J. 9(5):1665-1672). In this method, the wild-type or mutant LuxS cDNA may be expressed in a host organism on a growth medium depleted of either natural cysteine or methionine (or both) but enriched in selenocysteine or selenomethionine (or both). Alternatively, selenocysteine or selenomethionine mutants may be made using nonauxotrophic E. coli strains, e.g., by inhibiting methionine biosynthesis in these strains with high concentrations of Ile, Lys, Phe, Leu, Val or Thr and then providing selenomethionine in the medium (Doublié, 1997, Methods in Enzymology 276:523-530). Furthermore, selenocysteine can be selectively incorporated into polypeptides by exploiting the prokaryotic and eukaryotic mechanisms for selenocysteine incorporation into certain classes of LuxSs in vivo, as described in U.S. Pat. No. 5,700,660 to Leonard et al. (filed Jun. 7, 1995). One of skill in the art will recognize that selenocysteine is preferably not incorporated in place of cysteine residues that form disulfide bridges, as these may be important for maintaining the three-dimensional structure of the LuxS and are preferably not to be eliminated. One of skill in the art will further recognize that, in order to obtain accurate phase information, approximately one selenium atom should be incorporated for every 140 amino acid residues of the polypeptide chain. The number of selenium atoms incorporated into the polypeptide chain can be conveniently controlled by designing a Met or Cys mutant having an appropriate number of Met and/or Cys residues, as described more fully below.

In some instances, the polypeptide to be crystallized may not contain cysteine or methionine residues. Therefore, if selenomethionine and/or selenocysteine mutants are to be used to obtain heavy-atom derivative crystals, methionine and/or cysteine residues may be introduced into the polypeptide chain. Likewise, Cys residues must be introduced into the polypeptide chain if the use of a cysteine-binding heavy metal, such as mercury, is contemplated for production of a heavy-atom derivative crystal.

Such mutations are preferably introduced into the polypeptide sequence at sites that will not disturb the overall LuxS fold. For example, a residue that is conserved among many members of the LuxS family or that is thought to be involved in maintaining its activity or structural integrity, as determined by, e.g., sequence alignments, should not be mutated to a Met or Cys. In addition, conservative mutations, such as Ser to Cys, or Leu or Ile to Met, are preferably introduced. One additional consideration is that, in order for a heavy-atom derivative crystal to provide phase information for structure determination, the location of the heavy atom(s) in the crystal unit cell must be determinable and provide phase information. Therefore, a mutation is preferably not introduced into a portion of the LuxS that is likely to be mobile, e.g., at, or within about 1-5 residues of, the N- and C-termini.

Conversely, if there are too many methionine and/or cysteine residues in a polypeptide sequence, over-incorporation of the selenium-containing side chains can lead to the inability of the polypeptide to fold and/or crystallize, and may potentially lead to complications in solving the crystal structure. In this case, methionine and/or cysteine mutants are prepared by substituting one or more of these Met and/or Cys residues with another residue. The considerations for these substitutions are the same as those discussed above for mutations that introduce methionine and/or cysteine residues into the polypeptide. Specifically, the Met and/or Cys residues are preferably conservatively substituted with Leu/Ile and Ser, respectively.

As DNA encoding cysteine and methionine mutants can be used in the methods described above for obtaining SeCys and SeMet heavy-atom derivative crystals, the preferred Cys or Met mutant will have one Cys or Met residue for every 140 amino acids.

### 6.4 Production of Polypeptides

The native and mutated LuxS polypeptides described herein may be chemically synthesized in whole or part using techniques that are well-known in the art (see, e.g., Creighton, 1983, Proteins: Structures and Molecular Principles, W.H. Freeman & Co., NY.). Alternatively, methods that are well known to those skilled in the art can be used to construct expression vectors containing the native or mutated LuxS polypeptide coding sequence and appropriate transcriptional/translational control signals. These methods include in vitro recombinant DNA techniques, synthetic techniques and in vivo recombination/genetic recombination. See, for example, the techniques described in Maniatis et al., 1989, Molecular Cloning: A Laboratory Manual, Cold Spring Harbor Laboratory, NY and Ausubel et al., 1989, Current Protocols in Molecular Biology, Greene Publishing Associates and Wiley Interscience, NY.

A variety of host-expression vector systems may be utilized to express the LuxS coding sequence. These include, but are not limited to, microorganisms such as bacteria transformed with recombinant bacteriophage DNA, plasmid DNA or cosmid DNA expression vectors containing the LuxS coding sequence; yeast transformed with recombinant yeast expression vectors containing the LuxS coding sequence; insect cell systems infected with recombinant virus expression vectors (e.g., baculovirus) containing the LuxS coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the LuxS coding sequence; or animal cell systems. The expression elements of these systems vary in their strengths and specificities.

Specifically designed vectors allow the shuttling of DNA between hosts such as bacteria-yeast or bacteria-animal cells. An appropriately constructed expression vector may include: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

Depending on the host/vector system utilized, any of a number of suitable transcription and translation elements, including constitutive and inducible promoters, may be used in the expression vector. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the tyrosine kinase domain DNA, SV40-, BPV- and EBV-based vectors may be used with an appropriate selectable marker.

The expression vector may be introduced into host cells via any one of a number of techniques including but not limited to transformation, transfection, infection, protoplast fusion, and electroporation. The expression vector-containing cells are clonally propagated and individually analyzed to determine whether they produce LuxS. Identification of LuxS expressing host cell clones may be done by several means, including but not limited to immunological reactivity with anti-LuxS antibodies, and the presence of host cell-associated LuxS activity.

Expression of LuxS cDNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes.

To determine the LuxS cDNA sequence(s) that yields optimal levels of LuxS activity and/or protein LuxS, modified LuxS cDNA molecules are constructed. Host cells are transformed with the cDNA molecules and the levels of LuxS RNA and/or LuxS are measured.

Levels of LuxS in host cells are quantitated by a variety of methods such as immunoaffinity and/or ligand affinity techniques, LuxS-specific affinity beads or LuxS-specific antibodies are used to isolate ³⁵S-methionine labeled or unlabeled LuxS. Labeled or unlabeled LuxS is analyzed by SDS-PAGE. Unlabeled LuxS is detected by Western blotting, ELISA or RIA employing LuxS-specific antibodies. LuxS-specific antibodies can be obtained by techniques well known to those of skill in the art including, for instance, those disclosed in Harlow and Lane, Antibodies: A Laboratory Manual, Cold Spring Harbor Laboratory, 1988.

Following expression of LuxS in a recombinant host cell, LuxS may be recovered to provide LuxS in active form. Several protein purification procedures are available and suitable for use. Recombinant LuxS may be purified from cell lysates or from conditioned culture media, by various combinations of, or individual application of, fractionation, or chromatography steps that are known in the art.

In addition, recombinant LuxS can be separated from other cellular proteins by use of an immuno-affinity column made with monoclonal or polyclonal antibodies specific for full length nascent LuxS or polypeptide fragments thereof.

Alternatively, LuxS may be recovered from a host cell in an unfolded, inactive form, e.g., from inclusion bodies of bacteria. LuxSs recovered in this form may be solublized using a denaturant, e.g., guanidinium hydrochloride, and then refolded into an active form using methods known to those skilled in the art, such as dialysis.

### 6.5 Crystallization of LuxS Polypeptides and Characterization of Crystals

The native, heavy-atom derivative and co-crystals from which the atomic structure coordinates of the invention are obtained can be obtained by conventional means as are well-known in the art of protein crystallography, including batch, liquid bridge, dialysis, and vapor diffusion methods (see, e.g., McPherson, 1998, Crystallization of Biological Macromolecules, Cold Spring Harbor Press, New York; McPherson, 1990, Eur. J. Biochem. 189:1-23.; Weber, 1991, Adv. Protein Chem. 41:1-36.).

Generally, native crystals are grown by dissolving substantially pure LuxS polypeptide in an aqueous buffer comprising a precipitant at a concentration just below that necessary to precipitate the LuxS. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In a preferred embodiment, native crystals are grown by vapor diffusion in sitting drops (McPherson, 1982, Preparation and Analysis of Protein Crystals, John Wiley, New York; McPherson, 1990, Eur. J. Biochem. 189:1-23.). In this method, the polypeptide/precipitant solution is allowed to equilibrate in a closed container with a larger aqueous reservoir having a precipitant concentration optimal for producing crystals. Generally, less than about 25 μL of substantially pure polypeptide solution is mixed with an equal volume of reservoir solution, giving a precipitant concentration about half that required for crystallization. The sealed container is allowed to stand, usually for about 2-6 weeks, until crystals grow.

For native crystals from which the atomic structure coordinates of the invention are obtained, it has been found that sitting drops comprising about 1 μL of H. pylori LuxS polypeptide (5 mg/mL in 10 mM HEPES, pH 7.5, 150 mM sodium chloride, 10 mM methionine, 1 mM beta-mercaptoethanol) with 1 μL reservoir solution (32% w/v PEG 1000, 200 mM ammonium sulfate, and 100 mM MES, pH 5.75) suspended over 0.5 mL reservoir solution for about one week at 20° C. provide diffraction quality crystals. Similarly, sitting drops prepared by mixing about 1 μL of D. radiodurans LuxS polypeptide (19 mg/mL in 10 mM HEPES, pH 7.5, 150 mM sodium chloride, 10 mM methionine, 1 mM beta-mercaptoethanol) and 1 μL reservoir solution (26% w/v PEG monomethyl ether (“PEG MME”) 5000, and 100 mM MES, pH 6.5) suspended over 0.5 mL reservoir solution for about one week at 4° C. provide diffraction quality crystals. Sitting drops prepared by mixing about 1 μL of H. influenzae LuxS polypeptide (10 mg/mL in 10 mM HEPES, pH 7.5, 150 mM sodium chloride, 10 mM methionine, 1 mM beta-mercaptoethanol) and 1 μL reservoir solution (21% w/v PEG MME 5000, and 100 mM Bis-Tris, pH 6.25) suspended over 0.5 mL reservoir solution for about one week at 12° C. provide diffraction quality crystals.

Of course, those having skill in the art will recognize that the above-described crystallization conditions can be varied. Exemplary variations, which may be used alone or in combination include polypeptide solutions comprising polypeptide concentrations between about 3 mg/mL and about 25 mg/mL, buffer concentrations between about 5 mM and about 200 mM, sodium chloride concentrations between about 0 mM and about 400 mM, pH ranges between about 5.0 and about 7.0; and reservoir solutions comprising PEG or PEG MME concentrations between about 15% and about 35% (w/v), PEG or PEG MME average molecular weights between about 600 and about 10000, ammonium sulfate concentrations between about 0 mM and about 300 mM, and temperature ranges between 4° C. and 25° C. Any buffer solution capable of maintaining the desired pH range may be used, including, for example, ACES, ADA, BES, Bis-Tris Propane, Citric Acid, Imidazole, MOPS, PIPES, HEPES, MES, Tris, Bis-Tris and cacodylate.

Heavy-atom derivative crystals can be obtained by soaking native crystals in mother liquor containing salts of heavy metal atoms. Native crystals can be soaked with salts of heavy metal atoms according to methods known to those of skill in the art including, for instance, those disclosed in Stura and Chen, 1992, “Soaking of Crystals” in Crystallization of Nucleic Acids and Proteins: A Practical Approach, Ducruix and Giege eds., Oxford University Press. Exemplary mother liquor solutions include the sitting drop solutions described in detail above.

Heavy-atom derivative crystals can also be obtained from SeMet and/or SeCys mutants, as described above for native crystals.

Some mutant LuxSs may crystallize under slightly different crystallization conditions than wild-type LuxS, or under very different crystallization conditions, depending on the nature of the mutation, and its location in the LuxS. For example, a non-conservative mutation may result in alteration of the hydrophilicity of the mutant, which may in turn make the mutant LuxS either more soluble or less soluble than the wild-type LuxS. Typically, if a LuxS becomes more hydrophilic as a result of a mutation, it will be more soluble than the wild-type LuxS in an aqueous solution and a higher precipitant concentration will be needed to cause it to crystallize. Conversely, if a LuxS becomes less hydrophilic as a result of a mutation, it will be less soluble in an aqueous solution and a lower precipitant concentration will be needed to cause it to crystallize. If the mutation happens to be in a region of the LuxS involved in crystal lattice contacts, crystallization conditions may be affected in more unpredictable ways.

### 6.6 Characterization of LuxS Crystals

The dimensions of a unit cell of a crystal are defined by six numbers, the lengths of three unique edges, a, b, and c, and three unique angles, α, β, and γ. The type of unit cell that comprises a crystal is dependent on the values of these variables, as discussed above in Section 3.2.

When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the l index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections is recorded on the detector, resulting in a diffraction pattern as shown in FIG. 2.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average LuxS, and the typical solvent content of a LuxS crystal, which is usually in the range of 30-70% of the unit cell volume (Matthews, 1968, J. Mol. Biol. 33(2):491-497).

Several SeMet mLuxS crystals were obtained from different bacterial sources. The characteristics of these crystals are described below. The H. pylori LuxS crystals of the present invention are generally characterized by a diffraction pattern, as shown in FIG. 2. The crystals are further characterized by unit cell dimensions and space group symmetry information obtained from the diffraction patterns, as described above. The crystals, which may be native crystals, heavy-atom derivative crystals or co-crystals, have a tetragonal unit cell and space group symmetry P4₃2₁2. In one form of crystalline H. pylori LuxS, the unit cell has dimensions of a=71.04+/−0.7 Å, b=71.04+/−0.7 Å, c=130.14+/−1.3 Å. There are likely to be two LuxS molecules in the asymmetric unit, related by an approximate 2-fold axis. The crystals appear as long (up to 0.7 mm), thin (typically 0.05 to 0.1 mm wide) spikes.

LuxS crystals were also obtained from H. influenzae LuxS. The H. influenzaei LuxS crystals, which may be native crystals, heavy-atom derivative crystals or co-crystals, have a tetragonal unit cell and space group symmetry P4₂2₁2. In one form of crystalline H. influenzae LuxS, the unit cell has dimensions of a=129.59+/−1.3 Å, b=129.59+/−1.3 Å, c=53.74+/−0.5 Å. There are likely to be two LuxS molecules in the asymmetric unit, related by an approximate 2-fold axis. The crystals appear as long (up to 0.4 mm), thin (typically 0.05 to 0.1 mm wide) rods.

LuxS crystals of the invention were also obtained from D. radiodurans LuxS. In one form, the D. radiodurans LuxS crystals, which may be native crystals, heavy-atom derivative crystals or co-crystals, have a monoclinic unit cell and space group symmetry P2₁. The unit cell has dimensions of a=43.71+/−0.4 Å, b=82.18+/−0.8 Å, c=49.48+/−0.5 Å and beta=102.78+/−1.0 degrees. There are likely to be two LuxS molecules in the asymmetric unit, related by an approximate 2-fold axis. The crystals appear as small blocks (typically 0.05 to 0.1 mm on a side). In another form of D. radiodurans LuxS crystals, a C2 monoclinic unit cell is observed with dimensions of a=51.19+/−0.5 Å, b=70.14+/−0.7 Å, c=49.73+/−0.5 Å and beta=112.03+/−1.1 degree. There is one molecule of LuxS in the asymmetric unit.

### 6.7 Collection of Data and Determination of Structure Solutions

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal must be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire dataset from one crystal.

Once a dataset is collected, the information is used to determine the three-dimensional structure of the molecule in the crystal. However, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal. (Blundel et al., 1976, Protein Crystallography, Academic Press).

Another method of obtaining phase information is by molecular replacement, which is a method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal. (Lattman, 1985, Methods in Enzymology 115:55-77; Rossmann, 1972, “The Molecular Replacement Method,” Int. Sci. Rev. Ser. No. 13, Gordon & Breach, New York).

A third method of phase determination is multi-wavelength anomalous diffraction or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen et al., 2000, Acta Cryst., D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically reasonable conformation that fits the map precisely.

After a model is generated, a structure is refined. Refinement is the process of minimizing the function Φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function Φ converges on a minimum wherein the model fits the electron density map and is stereochemically and conformationally reasonable. During refinement, ordered solvent molecules are added to the structure.

### 6.8 Structures of LuxS

The present invention provides, for the first time, the high-resolution three-dimensional structure and atomic structure coordinates of crystalline LuxS as determined by X-ray crystallography. The specific methods used to obtain the structure coordinates are provided in the examples, infra. The atomic structure coordinates of four crystalline forms of LuxS are appended as Table 7, Table 8, Table 9, Table 10, Table 11, and Table 12 (H. pylori, H. influenzae, D. radiodurans P2₁, D. radiodurans C2, D. radiodurans C2, and D. radiodurans P1, respectively).

Those having skill in the art will recognize that atomic structure coordinates as determined by X-ray crystallography are not without error. Thus, it is to be understood that any set of structure coordinates obtained for crystals of LuxS, whether native crystals, heavy-atom derivative crystals or co-crystals, that have a root mean square deviation (“r.m.s.d.”) of less than or equal to about 2 Å when superimposed, using backbone atoms (N, Cα, C and O), on the structure coordinates listed in Table 7, Table 8, Table 9, Table 10, Table 11, and Table 12, are considered to be identical with the structure coordinates listed when at least about 50% to 100% contiguous or non-contiguous Cα atoms of LuxS are included in the superposition.

Referring now to FIG. 6, the overall structure of LuxS is of an “alpha-beta” fold, meaning one with approximately the same number of alpha helicies as beta strands. The various regions of the molecule are identified by residue in FIG. 1. For example, the designation “3333” in FIG. 1 indicates the residues of the 3/10 helix, discussed below. The four beta strands comprise an anti-parallel beta sheet, backed on one side by the three longest alpha helicies (#2, #3, and #4). Between the sheet and the helicies is the hydrophobic core of the polypeptide. A separate 3/10 helix is also observed at the N-terminal side of the protein. A striking coordination of three histidine residues (His 57, His 61, and His 137) and a cysteine residues (Cys 131) indicate a portion of the LuxS active site. Three of these residues (His 57, His 61, and Cys 131) are within coordination distance (less than 2.5 Å) of a metal ion obsereved in the experimental density map, which was determined to be zinc from EXAFS measurements on LuxS crystals (see FIG. 10A). This ion is clearly visible in the electron density map calculated from the phases obtained directly from the SHARP phasing program. As only three residues and not four are seen to coordinate with the metal it must have a lone pair of electrons available for whatever chemical processing the enzyme performs in its in vivo function.

Examination of residual density after modeling of the LuxS protein revealed a patch of density near the metal binding site. This was successfully modeled to be a methionine (see FIG. 11). This ligand was seen in both molecules in the asymmetric unit for H. pylori and H. influenzae and in one of the molecules (B) in D. radiodurans, space group P2₁. Methionine was present in the polypeptide solution used for crystallization in 10 mM concentration, added to keep Se atoms reduced. Thus there is no indication that methionine plays an in vivo role as a substrate for LuxS. Indeed, its sidechain is too short to reach the metal site (see FIG. 11). However, through modeling with the program SPOCK (Christopher, 1998, Texas A & M University) it is apparent that there is considerable room for a larger amino acid to bind in this region. When S-ribosylhomocysteine, a proposed substrate for LuxS (PCT WO 00/32152), was modeled into the amino acid binding site several highly conserved residues of LuxS were identified as significant due to their closed proximity to this ligand: Ser 9, His 14, Arg 23, Asp 40, Arg 42, Glu 60, Met 84, Cyc 86, Thr 88, and Tyr 91.

The recognition of the amino acid ligand is through its backbone atoms. The carbonyl group is within hydrogen bonding distance of the backbone amino proton of residue 81 (2.8 Å) and the amide group of the Arg68, Ile81 sidechain (3.1 Å). The amino group of the methionine ligand is within hydrogen bonding distance of residue 81 backbone carbonyl group (2.8 Å), and the sidechain carbonyl group of Asp 80 (2.6 Å). A water mediated hydrogen bond from the methionine carbonyl to the carbonyl of Glu 60 is also seen. Glu 60, Arg 68, and Asp 80 are highly conserved in the LuxS proteins (see FIG. 1) indicating that all possess a capacity to bind an amino acid or derivative thereof.

Van der Waal contacts of the methionine are made with both LuxS molecules in the homodimer (see below). The closest approaches to the methionine sidechain from nearby residues sidechain atoms are: 3.3 Å for Asp 80, 3.9 Å for Glu 60, 3.8 Å for Ala 64, and 3.8 Å for His 61, all in the same molecule that is binding the methionine backbone; and 3.8 Å for Tyr 91, 3.6 Å for Ser 9, 3.3 Å for Phe 10, and 3.5 Å for residue 7, all from the other molecule in the homodimer. The significance of these contacts are emphasized by the fact that all except residue 7 are highly conserved in the LuxS motif (see FIG. 1).

Three crystalline LuxS polypeptides displayed a homodimer interaction in their asymmetric units, and the fourth, the D. radiodurans C2 crystalline polypeptide, displayed a dimer with crystallographic symmetry. The dimerization is illustrated in FIG. 7. This dimerization was highly consistent between the three structures (alpha carbon superpositions of the dimers ranging from 1.0 Å² for D. radiodurans P2₁, onto H. influenzae to 1.2 Å² for D. radiodurans P2₁ onto H. pylori for residues 11-69, 77-118, and 125-152 of each monomer). The surface area buried through this interaction is 3930 Å² for the D. radiodurans P2₁, LuxS, 3,195 Å² for the D. radiodurans C2 crystallographic dimer, 4160 Å² for H. influenzae, and 4180 Å² for H. pylori. These are very significant, comprising nearly a quarter of each molecules surface. Thus we propose that LuxS functions as a homodimer in solution. More evidence for this is the fact that the methionine ligand binding occurs at the dimer interface (see FIG. 7B) and a channel is provided for ligand entrance and exit through the opposing molecule (see FIG. 12).

Yet more evidence is apparent from plots of electrostatic potential on the monomer surfaces (FIG. 13A) and plots of residue conservation (FIG. 13B). It is clear from FIG. 13A that there is a charge complementarity to the surfaces that interact to form the dimer. Also FIG. 13B demonstrate that the majority of conserved, non-hydrophobic residues line the dimerization domain, which is also the metal and amino acid binding domains, emphasizing its importance. Dynamic light scattering studies have given some support to the dimerization hypothesis. In particular, all four soluble proteins showed monodisperse dimer formation (apparent molecular weights of 1.8 to 2.1 times that of monomer).

A 1.65 Å resolution dataset of D. radiodurans LuxS purified in the absense of methionine, crystallized in the presence of Se-methionine, revealed oxidation of a cysteine residues (Cys82 in Table 12) near the active site (Example 6). Specifically, this residue has been modified to cysteinesulfinic acid through the addition of two oxygen atoms to the sidechain sulfur atom. This modification was present in the protein prior to crystallization and x-ray data collection as demonstrated by high resolution mass spectrometry measurements on the protein. The oxygens of the cysteine are involved in an extensive hydrogen-bonding network. Preliminary tests with the putative LuxS substrate, S-ribosylhomocystein (SRH), have shown no significant enzymatic activity. Other enzymes that possess this modification have been shown to be in an inactive state (for example K. Becker, et al., Nat. Struct. Biol. 5 (1998), 267-271). The unusual occurrence of this oxidation suggests a possible regulatory mechanism on the activity of LuxS.

A structure-based sequence alignment is shown in FIG. 1. The secondary structure assignments for LuxS were obtained using the Kabsch and Sander algorithm (Kabsch and Sander, 1983, “Dictionary of LuxS secondary structure: pattern recognition of hydrogen-bonded and geometrical features,” Biopolymers 22:2577-2637.) as implemented in PROCHECK (Laskowski et al., 1993, “PROCHECK: a program to check the stereochemical quality of LuxS structures,” J. Appl. Cryst. 26:283-291). In the LuxS sequences a hyphen denotes an insertion.

### 6.9 Atomic Structure Coordinates of LuxS

The molecular structure coordinates can be used in molecular modeling and design, as described more fully below. The present invention encompasses the structure coordinates and other information, e.g., amino acid sequence, connectivity tables, vector-based representations, temperature factors, etc., used to generate the three-dimensional structure of the polypeptide for use in the software programs described below and other software programs.

The invention encompasses machine readable media embedded with the three-dimensional structure of a crystalline polypeptide and/or model, such as, for example, its molecular structure coordinates, described herein, or with subunits, domains, and/or, portions thereof such as, for example, portions comprising active sites, accessory binding sites, and/or binding pockets in either liganded or unliganded forms. As used herein, “machine readable medium” refers to any medium that can be read and accessed directly by a computer or scanner. Such media may take many forms, including but not limited to, non-violatile, volatile and transmission media. Non-volatile media, i.e., media that can retain information in the absence of power, includes a ROM. Volatile media, i.e., media that can not retain information in the absence of power, includes a main memory. Transmission media includes coaxial cables, copper wire and fiber optics, including the wires that comprise the bus. Transmission media can also take the form of carrier waves; i.e., electromagnetic waves that can be modulated, as in frequency, amplitude or phase, to transmit information signals. Additionally, transmission media can take the form of acoustic or light waves, such as those generated during radio wave and infrared data communications.

Such media also include, but are not limited to: magnetic storage media, such as floppy discs, flexible discs, hard disc storage medium and magnetic tape; optical storage media such as optical discs or CD-ROM; electrical storage media such as RAM or ROM, PROM (i.e., programmable read only memory), EPROM (i.e., erasable programmable read only memory), including FLASH-EPROM, any other memory chip or cartridge, carrier waves, or any other medium from which a processor can retrieve information, and hybrids of these categories such as magnetic/optical storage media. Such media further include paper on which is recorded a representation of the molecular structure coordinates, e.g., Cartesian coordinates, that can be read by a scanning device and converted into a format readily accessed by a computer or by any of the software programs described herein by, for example, optical character recognition (OCR) software. Such media also include physical media with patterns of holes, such as, for example, punch cards, and paper tape.

A variety of data storage structures are available for creating a computer readable medium having recorded thereon the molecular structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, macromolecular Crystallographic Information File (“mmCIF”) and Protein Data Bank (“PDB”) format (Research Collaboratory for Structural Bioinformatics; http://www.rcsb.org; Cambridge Crystallographic Data Centre format (http://www.ccdc.cam.ac.uk/support/csd_doc/volume3/z323.html); Structure-data (“SD”) file format (MDL Information Systems, Inc.; Dalby et al., 1992, J. Chem. Inf. Comp. Sci. 32:244-255); and line-notation, e.g., as used in SMILES (Weininger, 1988, J. Chem. Inf. Comp. Sci. 28:31-36). Methods of converting between various formats read by different computer software will be readily apparent to those of skill in the art, e.g., BABEL (v. 1.06, Walters & Stahl, © 1992, 1993, 1994; http://www.brunel.ac.uk/departments/chem/babel.htm.) All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

A computer may be used to display the structure coordinates or the three-dimensional representation of the protein or peptide structures, or portions thereof, such as, for example, portions comprising active sites, accessory binding sites, and/or binding pockets, in either liganded or unliganded form, of the present invention. The term “computer” includes, but is not limited to, mainframe computers, personal computers, portable laptop computers, and personal data assistants (“PDAs”) which can store data and independently run one or more applications, i.e., programs. The computer may include, for example, a machine readable storage medium of the present invention, a working memory for storing instructions for processing the machine-readable data encoded in the machine readable storage medium, a central processing unit operably coupled to the working memory and to the machine readable storage medium for processing the machine readable information, and a display operably coupled to the central processing unit for displaying the structure coordinates or the three-dimensional representation. The information contained in the machine-readable medium may be in the form of, for example, X-ray diffraction data, structure coordinates, electron density maps, or ribbon structures. The information may also include such data for co-complexes between a compound and a protein or peptide of the present invention.

The computers of the present invention may preferably also include, for example, a central processing unit, a working memory which may be, for example, random-access memory (RAM) or “core memory,” mass storage memory (for example, one or more disk drives or CD-ROM drives), one or more cathode-ray tube (“CRT”) display terminals or one or more LCD displays, one or more keyboards, one or more input lines, and one or more output lines, all of which are interconnected by a conventional bidirectional system bus. Machine-readable data of the present invention may be inputted and/or outputted through a modem or modems connected by a telephone line or a dedicated data line (either of which may include, for example, wireless modes of communication). The input hardware may also (or instead) comprise CD-ROM drives or disk drives. Other examples of input devices are a keyboard, a mouse, a trackball, a finger pad, or cursor direction keys. Output hardware may also be implemented by conventional devices. For example, output hardware may include a CRT, or any other display terminal, a printer, or a disk drive. The CPU coordinates the use of the various input and output devices, coordinates data accesses from mass storage and accesses to and from working memory, and determines the order of data processing steps. The computer may use various software programs to process the data of the present invention, examples of many of these types of software are discussed throughout the present application.

Those of skill in the art will recognize that a set of structure coordinates is a relative set of points that define a shape in three dimensions. Therefore, two different sets of coordinates could define the identical or a similar shape. Also, minor changes in the individual coordinates may have very little effect on the peptide's shape. Minor changes in the overall structure may have very little to no effect, for example, on the binding pocket, and would not be expected to significantly alter the nature of compounds that might associate with the binding pocket.

Although Cartesian coordinates are important and convenient representations of the three-dimensional structure of a polypeptide, other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, and a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein in which an atom is located, and connectivity information, which indicates to which atoms a particular atom is bonded, is also useful for representing a three-dimensional molecular structure.

### 6.10 Uses of the Atomic Structure Coordinates

Structure information, typically in the form of molecular structure coordinates, can be used in a variety of computational or computer-based methods to, for example, design, screen for, and/or identify compounds that bind the crystallized polypeptide or a portion or fragment thereof, or to intelligently design mutants that have altered biological properties.

When designing or identifying compounds that may associate with a given protein, binding pockets are often analyzed. The term “binding pocket,” refers to a region of a protein that, because of its shape, likely associates with a chemical entity or compound. A binding pocket of a protein is usually involved in associating with the protein's natural ligands or substrates, and is often the basis for the protein's activity. Many drugs act by associating with the binding pocket of a protein. The binding pocket preferably comprises amino acid residues that line the cleft of the pocket. Those of ordinary skill in the art will recognize that the numbering system used for other isoforms of LuxS may be different, but that the corresponding amino acids may be determined with a homology software program known to those of ordinary skill in the art. More preferably, the binding pocket comprises amino acid residues Glu60, Arg68, Ile81, Asp80, Ala64, His61, Tyr91, Ser9, Phe10, Leu7, His14, Arg23, Asp40, Arg42, Met84, Cys86, and Thr88. A binding pocket homolog comprises amino acids having structure coordinates that have a root mean square deviation from structure coordinates, as indicated in FIG. 4, of the binding pocket amino acids of up to about 2.0 Å, preferably up to about 1.75 Å, preferably up to about 1.5 Å, preferably up to about 1.0 Å, and preferably up to about 0.75 Å.

Where a binding pocket or regulatory site is said to comprise amino acids having particular structure coordinates, the amino acids comprise the same amino acid residues, or may comprise amino acids having similar properties, as shown in, for example, Table 1, and have either the same relative three-dimensional structure coordinates as FIG. 4, or the group of amino acids residues named as part of the binding pocket have an rmsd of within 2 Å, preferably within 1.5 Å, preferably within 1.2 Å, preferably within 1 Å, preferably within 0.75 Å, and preferably within 0.5 Å of the structure coordinates of FIG. 4. Preferably, when comparing the structure coordinates of the backbone atoms of the amino acid residues, the rmsd is within 2 Å, preferably within 1.5 Å, preferably within 1.2 Å, preferably within 1 Å, preferably within 0.75 Å, and more preferably within 0.5 Å.

Software applications are available to compare structures, or portions thereof, to determine if they are sufficiently similar to the structures of the invention such as DALI (Holm and Sander, J. Mol. Biol. 233:123-38 (1993) (See European Bioinformatics Institute site at www.ebi.ac.uk/).; MOE; CE (Shindyalov, I N, Boume, P E (1998) “Protein Structure Alignment by Incremental Combinatorial Extension (CE) of the Optimal Path,” Protein Engineering, 11:739-47); and DEJAVU (Uppsala Software Factory; Kleywegt, G. S. & Jones, T. A., (1997) “Detecting Folding Motifs and Similarities in Protein Structure,” Methods in Enzymology, 277:525-45).

The crystals and structure coordinates obtained therefrom may be used for rational drug design to identify and/or design compounds that bind LuxS as an approach towards developing new therapeutic agents. For example, a high resolution X-ray structure of, for example, a crystallized protein saturated with solvent, will often show the locations of ordered solvent molecules around the protein, and in particular at or near putative binding sites on the protein. This information can then be used to design molecules that bind these sites, the compounds synthesized and tested for binding in biological assays. Travis, 1993, Science, 262:1374.

The structure may also be computationally screened with a plurality of molecules to determine their ability to bind to the LuxS at various sites. Such compounds can be used as targets or leads in medicinal chemistry efforts to identify, for example, inhibitors of potential therapeutic importance. Travis, 1993, Science 262:1374.

In still another embodiment, compounds can be developed that are analogues of natural substrates, reaction intermediates or reaction products of LuxS. The reaction intermediates of LuxS can be deduced from the substrates, or reaction products in co-complex with LuxS. The binding of substrates, reaction intermediates, and reaction products may change the conformation of the binding site, which provides additional information regarding binding patterns of potential ligands, activators, inhibitors, and the like. Such information is also useful to design improved analogues of known LuxS inhibitors or to design novel classes of inhibitors based on the substrates, reaction intermediates, and reaction products of LuxS and LuxS-inhibitor co-complexes. This provides a novel route for designing LuxS inhibitors with both high specificity and stability.

In yet another embodiment, the structure can be used to computationally screen small molecule data bases for chemical entities or compounds that can bind in whole, or in part, to LuxS. In this screening, the quality of fit of such entities or compounds to the binding site may be judged either by shape complementarity or by estimated interaction energy. Meng et al., 1992, J. Comp. Chem. 13:505-524.

Another method of screening or designing compounds that associate with a binding pocket includes, for example, computationally designing a negative image of the binding pocket. This negative image may be used to identify a set of pharmacaphores. A pharmacaphore is a description of functional groups and how they relate to each other in three-dimensional space. This set of pharmacaphores can be used to design compounds and screen chemical databases for compounds that match with the pharmacaphore. Compounds identified by this method may then be further evaluated computationally or experimentally for binding activity. Various computer programs may be used to create the negative image of the binding site, for example; GRID (Goodford, 1985, J. Med. Chem. 28:849-857); GRID is available from Oxford University, Oxford, UK); MCSS (Miranker & Karplus, 1991, Proteins: Structure, Function and Genetics 11:29-34; MCSS is available from Accelrys, Inc., San Diego, Calif.); LUDI (Bohm, 1992, J. Comp. Aid. Molec. Design 6:61-78; LUDI is available from Accelrys, Inc., San Diego, Calif.); DOCK (Kuntz et al.; 1982, J. Mol. Biol. 161:269-288; DOCK is available from University of California, San Francisco, Calif.); and MOE.

Thus, among the various embodiments of the present invention are methods of identifying, screening, and designing compounds that associate with a binding pocket or other binding site of LuxS.

The design of compounds that bind to and/or modulate LuxS, for example that inhibit or activate LuxS according to this invention generally involves consideration of two factors. First, the compound must be capable of physically and structurally associating, either covalently or non-covalently with LuxS. For example, covalent interactions may be important for designing irreversible or suicide inhibitors of a protein. Non-covalent molecular interactions important in the association of LuxS with the compound include hydrogen bonding, ionic interactions and van der Waals and hydrophobic interactions. Second, the compound must be able to assume a conformation that allows it to associate with LuxS. Although certain portions of the compound will not directly participate in this association with LuxS, those portions may still influence the overall conformation of the molecule and may have a significant impact on potency. Conformational requirements include the overall three-dimensional structure and orientation of the chemical group or compound in relation to all or a portion of the binding site, or the spacing between functional groups of a compound comprising several chemical groups that directly interact with LuxS. Lead compounds for the design of inhibitors may include, for example, furanones, and S-ribosyl homocysteine.

Computer modeling techniques may be used to assess the potential modulating or binding effect of a chemical compound on LuxS. If computer modeling indicates a strong interaction, the molecule may then be synthesized and tested for its ability to bind to LuxS and affect (by inhibiting or activating) its activity.

Modulating or other binding compounds of LuxS may be computationally evaluated and designed by means of a series of steps in which chemical groups or fragments are screened and selected for their ability to associate with the individual binding pockets or other areas of LuxS. Several methods are available to screen chemical groups or fragments for their ability to associate with LuxS. This process may begin by visual inspection of, for example, the active site on the computer screen based on the LuxS coordinates. Selected fragments or chemical groups may then be positioned in a variety of orientations, or docked, within an individual binding pocket of LuxS (Blaney, J. M. and Dixon, J. S., 1993, Perspectives in Drug Discovery and Design 1:301). Manual docking may be accomplished using software such as Insight II (Accelrys, San Diego, Calif.) MOE; CE (Shindyalov, I N, Bourne, P E (1998) “Protein Structure Alignment by Incremental Combinatorial Extension (CE) of the Optimal Path,” Protein Engineering, 11:739-47); and SYBYL (Molecular Modeling Software, Tripos Associates, Inc., St. Louis, Mo., 1992), followed by energy minimization and molecular dynamics with standard molecular mechanics force fields, such as CHARMM (Brooks, et al., 1983, J. Comp. Chem. 4:187-217); More automated docking may be accomplished by using programs such as DOCK (Kuntz et al., 1982, J. Mol. Biol. 161:269-288; DOCK is available from University of California, San Francisco, Calif.); AUTODOCK (Goodsell & Olsen, 1990, Proteins: Structure, Function, and Genetics 8:195-202; AUTODOCK is available from Scripps Research Institute, La Jolla, Calif.); GOLD (Cambridge Crystallographic Data Centre (CCDC); Jones et al., J. Mol. Biol. 245:43-53 (1995)); and FLEXX (Tripos, St. Louis, Mo.; Rarey, M., et al., J. Mol. Biol. 261:470-89 (1996)); AMBER (Weiner, et al., 1984, J. Am. Chem. Soc. 106: 765-84;) and C² MMFF (Merck Molecular Force Field; Accelrys, San Diego, Calif.).

Specialized computer programs may also assist in the process of selecting fragments or chemical groups. These include DOCK; GOLD; LUDI; FLEXX (Tripos, St. Louis, Mo.; Rarey, M., et al., J. Mol. Biol. 261:470-89 (1996)); and GLIDE (Eldridge, et al., J. Comput. Aided Mol. Des. 11:425-45 (1997); Schrödinger, Inc., Portland, Oreg.).

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of LuxS. This would be followed by manual model building using software such as SYBYL, (Tripos, St. Louis, Mo.); Insight II (Accelrys, San Diego, Calif.); and MOE (Chemical Computing Group, Inc., Montreal, Canada).

Useful programs to aid one of skill in the art in connecting the individual chemical groups or fragments include, for example:

1. CAVEAT (Bartlett et al., 1989, ‘CAVEAT: A Program to Facilitate the Structure-Derived Design of Biologically Active Molecules’. In Molecular Recognition in Chemical and Biological Problems’, Special Pub., Royal Chem. Soc. 78:182-196). CAVEAT is available from the University of California, Berkeley, Calif.;

2. 3D Database systems such as ISIS or MACCS-3D (MDL Information Systems, San Leandro, Calif.). This area is reviewed in Martin, 1992, J. Med. Chem. 35:2145-2154); and

3. HOOK (Eisen et al., 1994, Proteins: Struct., Funct., Genet., 19:199-221) (available from Accelrys, Inc., San Diego, Calif.);

4. LUDI (Bohm, 1992, J. Comp. Aid. Molec. Design 6:61-78). LUDI is available from Accelrys, Inc., San Diego, Calif.

Instead of proceeding to build a LuxS inhibitor in a step-wise fashion one fragment or chemical group at a time, as described above, LuxS binding compounds may be designed as a whole or ‘de novo’ using either an empty active site or optionally including some portion(s) of a known inhibitor(s). These methods include, for example:

1. LUDI (Bohm, 1992, J. Comp. Aid. Molec. Design 6:61-78). LUDI is available from Accelrys, Inc., San Diego, Calif.;

2. LEGEND (Nishibata & Itai, 1991, Tetrahedron 47:8985). LEGEND is available from Accelrys, Inc., San Diego, Calif.;

3. LeapFrog (available from Tripos, Inc., St. Louis, Mo.); and

4. SPROUT (Gillet et al., 1993, J. Comput. Aided Mol. Design 7:127-53) (available from the University of Leeds, U.K.)

5. GenStar (Murcko, M. A. and Rotstein, S. H. J. Comput. Aided Mol. Des. 7:23-43 (1993)).\

6. GroupBuild (Rotstein, S. H., and Murcko, M. A., J. Med. Chem. 36:1700 91993)).

7. GrowMol (Rich, D. H. et al., Chimia 51:45 (1997)).

8. Grow (UpJohn; Moon J, Howe W, Proteins 11: 314-328 (1991)).

9. SmoG (DeWitte, R. S., Abstr. Pap Am Chem. S. 214:6-Comp Part 1, Sep. 7, '997; DeWitte, R. S. & Shakhnovich, E. I., J. Am. Chem. Soc. 118:11733-44(1996)).

10. LigBuilder (PDB (www.rcsb.org/pdb); Wang R, Ying G, Lai L, J. Mol. Model. 6: 498-516 (1998)).

Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210; Balbes et al., 1994, Reviews in Computational Chemistry, 5:337-80 (Lipkowitz and Boyd, Eds.) (VCH, New York); Guida, 1994, Curr. Opin. Struct. Biol. 4:777-81.

During design and selection of compounds by the above methods, the efficiency with which that compound may bind to LuxS may be tested and optimized by computational evaluation. For example, a compound that has been designed or selected to function as a LuxS-inhibitor must also preferably occupy a volume not overlapping the volume occupied by the active site residues when the native substrate is bound, however, those of ordinary skill in the art will recognize that there is some flexibility, allowing for rearrangement of the side chains. An effective LuxS inhibitor must preferably demonstrate a relatively small difference in energy between its bound and free states (i.e., it must have a small deformation energy of binding and/or low conformational strain upon binding). Thus, the most efficient LuxS inhibitors should preferably be designed with a deformation energy of binding of not greater than 10 kcal/mol, preferably, not greater than 7 kcal/mol, more preferably, not greater than 5 kcal/mol, and more preferably not greater than 2 kcal/mol. LuxS inhibitors may interact with the protein in more than one conformation that is similar in overall binding energy. In those cases, the deformation energy of binding is taken to be the difference between the energy of the free compound and the average energy of the conformations observed when the inhibitor binds to the enzyme.

A compound selected or designed for binding to LuxS may be further computationally optimized so that in its bound state it would preferably lack repulsive electrostatic interaction with the target protein. Non-complementary electrostatic interactions include repulsive charge-charge, dipole-dipole and charge-dipole interactions. Specifically, the sum of all electrostatic interactions between the inhibitor and the protein when the inhibitor is bound to it preferably make a neutral or favorable contribution to the enthalpy of binding.

Specific computer software is available in the art to evaluate compound deformation energy and electrostatic interaction. Examples of programs designed for such uses include: Gaussian 94, revision C (Frisch, Gaussian, Inc., Pittsburgh, Pa. © 1995); AMBER, version 4.1 (Kollman, University of California at San Francisco, © 1995); QUANTA/CHARMM (Accelrys, Inc., San Diego, Calif., © 1995); Insight II/Discover (Accelrys, Inc., San Diego, Calif., © 1995); DelPhi (Accelrys, Inc., San Diego, Calif. © 1995); and AMSOL (Quantum Chemistry Program Exchange, Indiana University). These programs may be implemented, for instance, using a computer workstation, as are well-known in the art, for example, a LINUX, SGI or Sun workstation. Other hardware systems and software packages will be known to those skilled in the art.

Once a LuxS-binding compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to LuxS by the same computer methods described in detail above. Methods of structure-based drug design are described in, for example, Klebe, G., J. Mol. Med. 78:269-281 (2000); Hol. W. G. J., Angewandte Chemie (Int'l Edition in English) 25:767-852 (1986); and Gane, P. J. and Dean, P. M., Current Opinion in Structural Biology, 10:401-404 (2000).

Because LuxS may crystallize in more than one crystal form, the structure coordinates of LuxS, or portions thereof, are particularly useful to solve the structure of those other crystal forms of LuxS. They may also be used to solve the structure of LuxS mutants, LuxS co-complexes, or of the crystalline form of any other protein with significant amino acid sequence homology to any functional domain of LuxS.

Preferred homologs or mutants of LuxS have an amino acid sequence homology to the amino acid sequences of the invention of greater than 60%, more preferred proteins have a greater than 70% sequence homology, more preferred proteins have a greater than 80% sequence homology, more preferred proteins have a greater than 90% sequence homology, and most preferred proteins have greater than 95% sequence homology. A protein domain, region, or binding pocket may have a level of amino acid sequence homology to the corresponding domain, region, or binding pocket amino acid sequences of the invention of greater than 60%, more preferred proteins have a greater than 70% sequence homology, more preferred proteins have a greater than 80% sequence homology, more preferred proteins have a greater than 90% sequence homology, and most preferred proteins have greater than 95% sequence homology.

One method that may be employed for this purpose is molecular replacement. In this method, the unknown crystal structure, whether it is another crystal form of LuxS, a LuxS mutant, or a LuxS co-complex, or the crystal of some other protein with significant amino acid sequence homology to any functional domain of LuxS, may be determined using phase information from the LuxS structure coordinates. This method may provide an accurate three-dimensional structure for the unknown protein in the new crystal more quickly and efficiently than attempting to determine such information ab initio. In addition, in accordance with this invention, LuxS mutants may be crystallized in co-complex with known LuxS inhibitors. The crystal structures of a series of such complexes may then be solved by molecular replacement and compared with that of wild-type LuxS. Potential sites for modification within the various binding sites of the protein may thus be identified. This information provides an additional tool for determining the most efficient binding interactions, for example, increased hydrophobic interactions, between LuxS and a chemical group or compound.

If an unknown crystal form has the same space group as and similar cell dimensions to the known LuxS crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. For example, if the unknown crystal form is of a LuxS co-complex, then a difference electron density map between this map and the map derived from the native, uncomplexed crystal will ideally show only the electron density of the ligand. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined against data extending from about 500 Å to at least 3.0 Å and preferably 1.5 Å, until the refinement has converged to limits accepted by those skilled in the art, such as, but not limited to, R=0.2, Rfree=0.25. This may be determined using computer software, such as X-PLOR, CNX, or refmac (part of the CCP4 suite; Collaborative Computational Project, Number 4, 1994, “The CCP4 Suite: Programs for Protein Crystallography,” Acta Cryst. D50, 760-763). See, e.g., Blundell et al., 1976, Protein Crystallography, Academic Press.; Methods in Enzymology, Vols. 114 & 115, Wyckoff et al., eds., Academic Press, 1985; Methods in Enzymology, Vols. 276 and 277 (Carter & Sweet, eds., Academic Press 1997); “Application of Maximum Likelihood Refinement” G. Murshudov, A. Vagin and E. Dodson, (1996) in the Refinement of Protein Structures, Proceedings of Daresbury Study Weekend; G. N. Murshudov, A. A. Vagin and E. J. Dodson, (1997) Acta Cryst. D53, 240-255; G. N. Murshudov, A. Lebedev, A. A. Vagin, K. S. Wilson and E. J. Dodson (1999) Acta Cryst. section D55, 247-255. See, e.g., Blundell et al., 1976, Protein Crystallography, Academic Press.; Methods in Enzymology, Vols. 114 & 115, Wyckoff et al., eds., Academic Press, 1985; Methods in Enzymology, Vols. 276 and 277 (Carter & Sweet, eds., Academic Press 1997). This information may thus be used to optimize known classes of LuxS inhibitors, and more importantly, to design and synthesize novel classes of LuxS inhibitors.

The structure coordinates of LuxS mutants will also facilitate the identification of related proteins or enzymes analogous to LuxS in function, structure or both, thereby further leading to novel therapeutic modes for treating or preventing LuxS mediated diseases.

Subsets of the molecular structure coordinates can be used in any of the above methods. Particularly useful subsets of the coordinates include, but are not limited to, coordinates of single domains, coordinates of residues lining an active site, coordinates of residues that participate in important protein-protein contacts at an interface, and alpha-carbon coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain, although useful for many applications, do not necessarily need to be used for the methods described herein.

### 7. EXAMPLE 1

### Preparation Of Crystals Of H. pylori LuxS Protein

The subsections below describe the production of a polypeptide containing the H. pylori LuxS protein, and the preparation and characterization of diffraction quality crystals, heavy-atom derivative crystals.

7.1 Production and Purification of LuxS

7.1.1 Preparation of LuxS Native Crystals

An open-reading frame for LuxS was amplified from H. pylori (Hp-ATCC 43504D) genomic DNA by the polymerase chain reaction (PCR) using the following primers:

Forward primer:
GGATTTCACATATGAAAATGAAT GTAGAGAGTTTC
Reverse Primer:
GTTCGGATCCAACCCCCACTTCAGACC

The PCR product (456 base pairs expected) is digested with NdeI and BamHI following the manufacturers' instructions, electrophoresed on a 1% agarose gel in TBE buffer and the appropriate size band is excised from the gel and eluted using a standard gel extraction kit. The eluted DNA is ligated overnight with T4 DNA ligase at 16° C. into pSB3, previously digested with NdeI and BamHI. The vector pSB3 is a modified version of pET26b (Novagen, Madison, Wis.) wherein the following sequence has been inserted into the BamHI site: GGATCCCACCACCACCACCACCACTGAGATCC. The resulting sequence of the gene after being ligated into the vector, from the Shine-Dalgarno sequence through the stop site and the “original” BamHI, site is as follows: AAGGAGGAGATATACATATG[ORF]GGATCCCACCACCACCACCACCACTGAG ATCC. The LuxS expressed using this vector has 8 amino acids added to the C-terminal end (GlySerHisHisHisHisHisHis).

Plasmids containing ligated inserts were transformed into chemically competent E. coli, such as Top 10 cells. Colonies were then screened for inserts in the correct orientation and miniprepped. The miniprep DNA was transformed into BL21 (DE3) Active Motif cells and plated onto petri dishes containing LB agar with 30 μg/ml of kanamycin. Isolated, single colonies were grown to mid-log phase and stored at −80° C. in LB containing 15% glycerol.

LuxS containing selenomethionine is overexpressed in E. coli by the addition of 200 μl 1M IPTG per 500 ml culture of minimal broth plus selenomethionine, and the cultures are allowed to ferment overnight. The LuxS was purified as follows. Cells were collected by centrifugation, lysed in cracking buffer, (50 mM Tris-HCl (pH 7.8), 500 mM NaCl, 10 mM imidazole, 10 mM methionine, 10% glycerol) and centrifuged to remove cell debris. The soluble fraction was purified over an IMAC column charged with nickel (Pharmacia, Uppsala, Sweden), and eluted under native conditions with imidazole. The protein was then further purified by gel filtration using a Superdex 75 column into 10 mM HEPES, pH 7.5 10 mM methionine, 1 mM βME, 150 mM NaCl, at a protein concentration of approximately 3 to 30 mg.

7.2 Crystallization Conditions

For crystals of H. pylori LuxS from which the molecular structure coordinates of the invention are obtained, it has been found that a hanging drop containing 1 μl of LuxS polypeptide 5 mg/mL in 10 mM HEPES pH7.5, 150 mM NaCl, 1 mM βME, 10 mM methionine, and 1 μl reservoir solution: 32% (w/v) PEG1000, 200 mM ammonium sulfate, 2 mM β-mercaptoethanol, and 100 mM MES, pH 5.75 in a sealed container containing 500 μL reservoir solution, incubated for 3-7 days at 20° C. provide diffraction quality crystals.

Other preferred methods of obtaining a crystal comprise the steps of:(a) mixing a volume of a solution comprising the LuxS with a volume of a reservoir solution comprising a precipitant, such as, for example, polyethylene glycol; and (b) incubating the mixture obtained in step (a) over the reservoir solution in a closed container, under conditions suitable for crystallization until the crystal forms. At least 27% (w/v) of PEG 1000 is present in the reservoir solution. PEG 1000 is preferably present in a concentration up to about 37% (w/v). Most preferably the concentration of PEG 1000 is 32% (w/v). The concentration of MES is preferably at least 10 mM. The concentration of MES is preferably up to about 200 mM. Most preferably, the concentration of MES is 100 mM. The concentration of ammonium sulfate is preferably at least 10 mM. The concentration of ammonium sulfate is preferably up to about 400 mM. The concentration of ammonium sulfate is most preferably 200 mM. For preferred crystallization conditions, the reservoir solution has a pH of at least 5.25. Preferably, the reservoir solution has a pH up to about 6.25. Most preferably, the pH is about 5.75. In preferred crystallization conditions, the temperature is at least 12° C. It is also preferred that the temperature is up to about 28° C. Most preferably, the temperature is 20° C.

Those of ordinary skill in the art recognize that the drop and reservoir volumes may be varied within certain biophysical conditions and still allow crystallization.

7.2.1 Preparation of Seleno-methionine Crystals

LuxS with seleno-methionine substitution was prepared in the same manner as for the native crystals except a minimum broth with seleno-methionine as the sole source of methionine was employed.

7.3 Analysis and Characterization of LuxS Crystals

7.3.1 Diffraction Data Collection

The crystals were individually harvested from their trays and transferred to a cryoprotectant consisting of 80% well solution, 10% glycerol, and 10% ethylene glycol. After about 30 seconds the crystal was collected and frozen in a stream of nitrogen gas that had been cooled to 95 degrees Kelvin. The crystals were then transferred in liquid nitrogen to the Advanced Photon Source (Argonne National Laboratory) where a two wavelength MAD experiment was collected, a peak wavelength and a high energy remote wavelength.

7.3.2 Structure Determination

The MAD data was indexed and intergrated using the program Denzo and merged and scaled using the program Scalepack (Otwinowski, A., and Minor, W. (1997) Methods Enzymol. 276:307-326). The program SnB (Weeks, C. M. and Miller, R., (1999) Acta Cryst. D55, 492-500) was then used to determine the location of Selenium-methionine Se's based on the peak wavelength data. These Se sites (10 of 12 Se were identified) were refined and phase information for the protein obtained using the program SHARP. Solomon solvent flattening of the data was subsequently employed in SHARP (LaFortelle, E. and Bricogne, G. (1997) Methods Enzymol. 276:472-94. The resulting map was viewed in the program O (Jones, T. A., and Kjeldgaard, M., (1997) Methods Enzymol. 277:173-208) and found to be of excellent quality with essentially all of both of the proteins in the asymmetric unit, main chain and sidechains, easily visible. This map was modeled using O to give the position of nearly all of the residues: residues 3 through 160 (molecule A) and 3 through 161 (molecule B) of H. pylori LuxS. The model was refined using the program CNX (Brunger et al., Acta Cryst. D53, 240-55; Accelrys (2000) Crystallography and NMR Explorer 2000.1).

7.3.3 Structure Analyses

The stereochemical quality of the atomic model was monitored using PROCHECK (Laskowski et al., 1993, J. Appl. Cryst. 26:283-291). As defined in PROCHECK, for H. pylori LuxS, there are 87.7% (molecule A) and 86.2% (molecule B) of the residues in the model have main-chain torsion angles in the most favored Ramachandran regions . No residues fall in the disallowed region. In H. pylori, there are only two residues of molecule A and none of molecule B that fall in the generously allowed regions. The overall G-factor scores are 0.16 (H. pylori, molecule A) and 0.13 (H. pylori, molecule B).

### 8. EXAMPLE 2

### Preparation of Crystals Of H. influenzae LuxS Protein

The subsections below describe the production of a polypeptide containing the H. influenzae LuxS protein, and the preparation and characterization of diffraction quality crystals, heavy-atom derivative crystals.

8.1 Production and Purification of LuxS

8.1.1 Preparation of LuxS Native Crystals

An open-reading frame for LuxS was amplified from H. influenzae (Hi-ATCC 51907D) genomic DNA by the polymerase chain reaction (PCR) using the following primers:

| | |
|---|---|
| Forward primer: | GGATTTCACATATGCCATTACTTGATAG |
| Reverse Primer | GTTCGGATCCTTTTAATAAGGAATTATCG |

The PCR product (501 base pairs expected) is digested with NdeI and BamHI following the manufacturers' instructions, electrophoresed on a 1% agarose gel in TBE buffer and the appropriate size band is excised from the gel and eluted using a standard gel extraction kit. The eluted DNA is ligated overnight with T4 DNA ligase at 16° C. into pSB3, previously digested with NdeI and BamHI. The vector pSB3 is a modified version of pET26b (Novagen, Madison, Wis.) wherein the following sequence has been inserted into the BamHI site: GGATCCCACCACCACCACCACCACTGAGATCC. The resulting sequence of the gene after being ligated into the vector, from the Shine-Dalgarno sequence through the stop site and the “original” BamHI, site is as follows: AAGGAGGAGATATACATATG[ORF]GGATCCCACCACCACCACCACCACTGAG ATCC. The LuxS expressed using this vector has 8 amino acids added to the C-terminal end (GlySerHisHisHisHisHisHis).

Plasmids containing ligated inserts were transformed into chemically competent E. coli, such as Top 10 cells. Colonies were then screened for inserts in the correct orientation and miniprepped. The miniprep DNA was transformed into BL21 (DE3) Active Motif cells and plated onto petri dishes containing LB agar with 30 μg/ml of kanamycin. Isolated, single colonies were grown to mid-log phase and stored at −80° C. in LB containing 15% glycerol.

LuxS containing selenomethionine is overexpressed in E. coli by the addition of 200 μl M IPTG per 500 ml culture of minimal broth plus selenomethionine, and the cultures are allowed to ferment overnight. The LuxS was purified as follows. Cells were collected by centrifugation, lysed in cracking buffer, (50 mM Tris-HCl (pH 7.8), 500 mM NaCl, 10 mM imidazole, 10 mM methionine, 10% glycerol) and centrifuged to remove cell debris. The soluble fraction was purified over an IMAC column charged with nickel (Pharmacia, Uppsala, Sweden), and eluted under native conditions with imidazole. The protein was then further purified by gel filtration using a Superdex 75 column into 10 mM HEPES, 10 mM methionine, 150 mM NaCl, at a protein concentration of approximately 3 to 30 mg.

8.2 Crystallization Conditions

For crystals of H. influenzae LuxS from which the molecular structure coordinates of the invention are obtained, it has been found that a hanging drop containing 1 μl of LuxS polypeptide 10 mg/mL in 10 mM HEPES pH 7.5, 150 mM NaCl, 1 mM βME 10 mM methionine, and 1 μl reservoir solution: 21% (w/v) PEG MME 5000, 2mM β-mercaptoethanol, and 100 mM Bis-Tris, pH 6.25 in a sealed container containing 500 μL reservoir solution, incubated for 3 days at 12° C. provide diffraction quality crystals.

Other preferred methods of obtaining a crystal comprise the steps of:(a) mixing a volume of a solution comprising the LuxS with a volume of a reservoir solution comprising a precipitant, such as, for example, polyethylene glycol; and (b) incubating the mixture obtained in step (a) over the reservoir solution in a closed container, under conditions suitable for crystallization until the crystal forms. At least 16% (w/v) of PEG MME 5000 is present in the reservoir solution. PEG MME 5000 is preferably present in a concentration up to about 27% (w/v). Most preferably the concentration of PEG MME 5000 is 21% (w/v). The concentration of Bis-Tris is preferably at least 10 mM. The concentration of Bis-Tris is preferably up to about 200 mM. Most preferably, the concentration of Bis-Tris is 100 mM. For preferred crystallization conditions, the reservoir solution has a pH of at least 5.75. Preferably, the reservoir solution has a pH up to about 6.75. Most preferably, the pH is about 6.25. In preferred crystallization conditions, the temperature is at least 4° C. It is also preferred that the temperature is up to about 20° C. Most preferably, the temperature is 12° C.

Those of ordinary skill in the art recognize that the drop and reservoir volumes may be varied within certain biophysical conditions and still allow crystallization.

8.2.1 Preparation of Seleno-methionine Crystals

LuxS with seleno-methionine substitution was prepared in the same manner as for the native crystals except a minimum broth with seleno-methionine as the sole source of methionine was employed.

8.3 Analysis and Characterization of LuxS Crystals

8.3.1 Diffraction Data Collection

The crystals were individually harvested from their trays and transferred to a cryoprotectant consisting of 80% well solution, 10% glycerol, and 10% ethylene glycol. After about 30 seconds the crystal was collected and frozen in a stream of nitrogen gas that had been cooled to 95 degrees Kelvin. The crystals were then transferred in liquid nitrogen to the Advanced Photon Source (Argonne National Laboratory) where a two wavelength MAD experiment was collected, a peak wavelength and a high energy remote wavelength.

8.3.2 Structure Determination

The MAD data was indexed and integrated using the program Denzo and merged and scaled using the program Scalepack. The program SnB was then used to determine the location of Selenium-methionine Se's based on the peak wavelength data. These Se sites (12 of 14 were found) were refined and phase information for the protein obtained using the program SHARP. Solomon solvent flattening of the data was subsequently employed in SHARP. The resulting map was viewed in the program O and found to be of excellent quality with essentially all of both of the proteins in the asymmetric unit, main chain and sidechains, easily visible. This map was modeled using O to give the position of nearly all of the residues: residues 6 through 164 (molecule A) and 6 through 166 (molecule B) of H. influenzae LuxS. The model was refined using the program CNX.

8.3.3 Structure Analyses

The stereochemical quality of the atomic model was monitored using PROCHECK (Laskowski et al., 1993, “PROCHECK: a program to check the stereochemical quality of LuxS structures,” J. Appl. Cryst. 26:283-291). As defined in PROCHECK, for H. influenzae LuxS there are 92.8% (molecule A) and 90.8% (molecule B) of the residues in the model have main-chain torsion angles in the most favored Ramachandran regions . There is only one residue that falls in the disallowed region (H. Influenzae, molecule B). H. influenzae has one residue of each molecule falling in the generously allowed regions. The overall G-factor scores are 0.25 (H. influenzae, molecules A and B).

### 9. EXAMPLE 3

### Preparation of P2₁ Crystals of D. radiodurans LuxS Protein

The subsections below describe the production of a polypeptide containing the D. radiodurans LuxS protein, and the preparation and characterization of diffraction quality crystals, heavy-atom derivative crystals.

9.1 Production and Purification of LuxS

9.1.1 Preparation of LuxS Native Crystals

An open-reading frame for LuxS was amplified from D. radiodurans (Dr-ATCC 13939D) genomic DNA by the polymerase chain reaction (PCR) using the following primers:

| | |
|---|---|
| Forward primer: | GGATTTCACATATGCCCGACATGGCAAACGTC |
| Reverse Primer: | GTTCGGATCCGCGTTCGAGCAGAATGGTTTC |

The PCR product (474 base pairs expected) is digested with NdeI and BamHI following the manufacturers' instructions, electrophoresed on a 1% agarose gel in TBE buffer and the appropriate size band is excised from the gel and eluted using a standard gel extraction kit. The eluted DNA is ligated overnight with T4 DNA ligase at 16° C. into pSB3, previously digested with NdeI and BamHI. The vector pSB3 is a modified version of pET26b (Novagen, Madison, Wis.) wherein the following sequence has been inserted into the BamHI site: GGATCCCACCACCACCACCACCACTGAGATCC. The resulting sequence of the gene after being ligated into the vector, from the Shine-Dalgarno sequence through the stop site and the “original” BamHI, site is as follows: AAGGAGGAGATATACATATG[ORF]GGATCCCACCACCACCACCACCACTGAG ATCC. The LuxS expressed using this vector has 8 amino acids added to the C-terminal end (GlySerHisHisHisHisHisHis).

Plasmids containing ligated inserts were transformed into chemically competent E. coli, such as Top 10 cells. Colonies were then screened for inserts in the correct orientation and miniprepped. The miniprep DNA was transformed into BL21 (DE3) pLysS Invitrogen cells and plated onto petri dishes containing LB agar with 30 μg/ml of kanamycin. Isolated, single colonies were grown to mid-log phase and stored at −80° C. in LB containing 15% glycerol.

LuxS containing selenomethionine is overexpressed in E. coli by the addition of 200 μl 1M IPTG per 500 ml culture of minimal broth plus selenomethionine, and the cultures are allowed to ferment overnight. The LuxS was purified as follows. Cells were collected by centrifugation, lysed in cracking buffer, (50 mM Tris-HCl (pH 7.8), 500 mM NaCl, 10 mM imidazole, 10 mM methionine, 10% glycerol) and centrifuged to remove cell debris. The soluble fraction was purified over an IMAC column charged with nickel (Pharmacia, Uppsala, Sweden), and eluted under native conditions with imidazole. The protein was then further purified by gel filtration using a Superdex 75 column into 10 mM HEPES, 10 mM methionine, 150 mM NaCl, at a protein concentration of approximately 3 to 30 mg.

9.2 Crystallization Conditions

For crystals of D. radiodurans LuxS from which the molecular structure coordinates of the invention are obtained, it has been found that a hanging drop containing 1 μl of LuxS polypeptide 19 mg/mL in 10 mM HEPES pH 7.5, 150 mM NaCl, 1 mM βME, 10 mM methionine, and 1l reservoir solution: 26% (w/v), PEG MME 5,000, 2 mM β-mercaptoethanol, and 100 mM MES, pH 6.5 in a sealed container containing 500 μL reservoir solution, incubated for one day at 4° C. provide diffraction quality crystals.

Other preferred methods of obtaining a crystal comprise the steps of:(a) mixing a volume of a solution comprising the LuxS with a volume of a reservoir solution comprising a precipitant, such as, for example, polyethylene glycol; and (b) incubating the mixture obtained in step (a) over the reservoir solution in a closed container, under conditions suitable for crystallization until the crystal forms. At least 21% (w/v) PEG MME 5,000 is present in the reservoir solution. PEG MME 5,000 is preferably present in a concentration up to about 31% (w/v). Most preferably the concentration of PEG MME 5,000 is 26% (w/v). The concentration of MES is preferably at least 10 mM. The concentration of MES is preferably up to about 200 mM. Most preferably, the concentration of MES is 100 mM. For preferred crystallization conditions, the reservoir solution has a pH of at least 6.0. Preferably, the reservoir solution has a pH up to about 7.0. Most preferably, the pH is about 6.5. In preferred crystallization conditions, the temperature is at least 0° C. It is also preferred that the temperature is up to about 12° C. Most preferably, the temperature is 4° C.

Those of ordinary skill in the art recognize that the drop and reservoir volumes may be varied within certain biophysical conditions and still allow crystallization.

9.2.1 Preparation of Seleno-methionine Crystals

LuxS with seleno-methionine substitution was prepared in the same manner as for the native crystals except a minimum broth with seleno-methionine as the sole source of methionine was employed.

9.3 Analysis and Characterization of LuxS Crystals

9.3.1 Diffraction Data Collection

The crystals were individually harvested from their trays and transferred to a cryoprotectant consisting of 80% well solution, 10% glycerol, and 10% ethylene glycol. After about 30 seconds the crystal was collected and frozen in a stream of nitrogen gas that had been cooled to 95 degrees Kelvin. The crystals were then transferred in liquid nitrogen to the Advanced Photon Source (Argonne National Laboratory) where a two wavelength MAD experiment was collected, a peak wavelength and a high energy remote wavelength.

9.3.2 Structure Determination

The MAD data was indexed and intergrated using the program Denzo and merged and scaled using the program Scalepack. The program SnB was then used to determine the location of Selenium-methionine Se's based on the peak wavelength data. These Se sites (11 of 14 were seen) were refined and phase information for the protein obtained using the program SHARP. Solomon solvent flattening of the data was subsequently employed in SHARP. The resulting map was viewed in the program O and found to be of excellent quality with essentially all of both of the proteins in the asymmetric unit, main chain and sidechains, easily visible. This map was modeled using O to give the position of nearly all of the residues: residues 6 through 162 (molecule A) and 8 through 162 (molecule B) of D. radiodurans LuxS. The model was refined using the program CNX.

9.3.3 Structure Analyses

The stereochemical quality of the atomic model was monitored using PROCHECK (Laskowski et al., 1993, “PROCHECK: a program to check the stereochemical quality of LuxS structures,” J. Appl. Cryst. 26:283-291). As defined in PROCHECK, D. radiodurans LuxS has 92.2% (molecule A) and 89.7% (molecule B) of the residues in the model have main-chain torsion angles in the most favored Ramachandran regions. There were no residues that fell in the disallowed region, nor any that fell in the generously allowed regions. The overall G-factor scores are 0.15 for both molecules A and B).

### 10. EXAMPLE 4

### Preparation of C2 Crystals of D. radiodurans LuxS Protein

The subsections below describe the production of a polypeptide containing the D. radiodurans LuxS protein, and the preparation and characterization of diffraction quality crystals, heavy-atom derivative crystals.

10.1 Production and Purification of LuxS

10.1.1 Preparation of LuxS Native Crystals

LuxS was overexpressed in E. coli cells as in Example 3.

The LuxS was purified as follows. Cells were collected by centrifugation, lysed in cracking buffer, (50 mM Tris-HCl (pH 7.8), 500 mM NaCl, 10 mM imidazole, 10 mM methionine, 10% glycerol) and centrifuged to remove cell debris. The soluble fraction was purified over an IMAC column charged with nickel (Pharmacia, Uppsala, Sweden), and eluted under native conditions with imidazole. The protein was then further purified by gel filtration using a Superdex 75 column into 10 mM HEPES, 10 mM methionine, 150 mM NaCl, at a protein concentration of approximately 3 to 30 mg.

10.2 Crystallization Conditions

For crystals of D. radiodurans LuxS from which the molecular structure coordinates of the invention are obtained, it has been found that a hanging drop containing 1 l of LuxS polypeptide 19 mg/mL in 10 mM HEPES pH 7.5, 150 mM NaCl, 1 mM βME, 10 mM methionine, and 1 μl reservoir solution: 26% (w/v) PEG MME 5,000, 2mM β-mercaptoethanol, and 100 mM MES, pH 6.5, in a sealed container containing 500 μL reservoir solution, incubated for one day at 4° C. provide diffraction quality crystals.

Other preferred methods of obtaining a crystal comprise the steps of:(a) mixing a volume of a solution comprising the LuxS with a volume of a reservoir solution comprising a precipitant, such as, for example, polyethylene glycol; and (b) incubating the mixture obtained in step (a) over the reservoir solution in a closed container, under conditions suitable for crystallization until the crystal forms. At least 21% (w/v) of PEG MME 5,000 is present in the reservoir solution. PEG MME 5,000 is preferably present in a concentration up to about 31% (w/v). Most preferably the concentration of PEG MME 5,000 is 26% (w/v). The concentration of MES is preferably at least 10 mM. The concentration of MES is preferably up to about 200 mM. Most preferably, the concentration of MES is 100 mM. For preferred crystallization conditions, the reservoir solution has a pH of at least 6.0. Preferably, the reservoir solution has a pH up to about 7.0. Most preferably, the pH is about 6.5. In preferred crystallization conditions, the temperature is at least 0° C. It is also preferred that the temperature is up to about 12° C. Most preferably, the temperature is 4° C.

Those of ordinary skill in the art recognize that the drop and reservoir volumes may be varied within certain biophysical conditions and still allow crystallization.

10.2.1 Preparation of Seleno-methionine Crystals

LuxS with seleno-methionine substitution was prepared in the same manner as for the native crystals except a minimum broth with seleno-methionine as the sole source of methionine was employed.

10.3 Analysis and Characterization of LuxS Crystals

10.3.1 Diffraction Data Collection

The crystals were individually harvested from their trays and transferred to a cryoprotectant consisting of 80% well solution, 10% glycerol, and 10% ethylene glycol. After about 30 seconds the crystal was collected and frozen in a stream of nitrogen gas that had been cooled to 95 degrees Kelvin. The crystals were then transferred in liquid nitrogen to the Advanced Photon Source (Argonne National Laboratory) where a two wavelength MAD experiment was collected, a peak wavelength and a high energy remote wavelength.

10.3.2 Structure Determination

This data was not sufficient on its own to solve the structure. However, the LuxS model from the P2₁ structure from D. radiodurans was used along with the molecular replacement program EPMR to give a solution using the high energy remote data calculated to average anomalous differences. The resulting map was viewed in the program O and found to be of excellent quality with essentially all of both of the proteins in the asymmetric unit, main chain and sidechains, easily visible. This map was modeled using O to give the position of nearly all of the residues: residues 12 through 162. The model was refined using the program CNX.

10.3.3 Structure Analyses

The stereochemical quality of the atomic model was monitored using PROCHECK (Laskowski et al., 1993, “PROCHECK: a program to check the stereochemical quality of LuxS structures,” J. Appl. Cryst. 26:283-291). As defined in PROCHECK, 91.8% of the residues in the model of D. radiodurans C2 LuxS have main chain torsion angles in the most favored Ramachandran regions. No residues fell in the disallowed region, and no residues fell in the generously allowed regions. The overall G-factor score is 0.23.

### 11. EXAMPLE 5

### Preparation of Crystals of D. radiodurans LuxS Protein in the Absence of Methionine

LuxS from D. radiodurans was expressed and purified in essentially the same manner as in Example 4, with the exception that the gel filtration buffer did not contain 10 mM methionine.

11.1 Crystallization Conditions

For crystals of D. radiodurans LuxS from which the molecular structure coordinates of the invention are obtained, it has been found that a hanging drop containing 1 μl of LuxS polypeptide 19 mg/mL in 10 mM HEPES pH 4.5, 150 mM NaCl, 1 mM βME, and 1 μl reservoir solution: 26% (w/v) PEG MME 5,000, 2 mM β-mercaptoethanol, and 100 mM MES, pH 6.5, in a sealed container containing 500 μL reservoir solution, incubated for one day at 4° C. provide diffraction quality crystals.

Other preferred methods of obtaining a crystal comprise the steps of:(a) mixing a volume of a solution comprising the LuxS with a volume of a reservoir solution comprising a precipitant, such as, for example, polyethylene glycol; and (b) incubating the mixture obtained in step (a) over the reservoir solution in a closed container, under conditions suitable for crystallization until the crystal forms. At least 21% (w/v) of PEG MME 5,000 is present in the reservoir solution. PEG MME 5,000 is preferably present in a concentration up to about 31% (w/v). Most preferably the concentration of PEG MME 5,000 is 26% (w/v). The concentration of MES is preferably at least 10 mM. The concentration of MES is preferably up to about 200 mM. Most preferably, the concentration of MES is 100 mM. For preferred crystallization conditions, the reservoir solution has a pH of at least 6.0. Preferably, the reservoir solution has a pH up to about 7.0. Most preferably, the pH is about 6.5. In preferred crystallization conditions, the temperature is at least 0° C. It is also preferred that the temperature is up to about 12° C. Most preferably, the temperature is 4° C.

Those of ordinary skill in the art recognize that the drop and reservoir volumes may be varied within certain biophysical conditions and still allow crystallization.

11.2. Preparation of Seleno-methionine Crystals

LuxS with seleno-methionine substitution was prepared in the same manner as for the native crystals except a minimum broth with seleno-methionine as the sole source of methionine was employed.

11.3 Analysis and Characterization of LuxS Crystals

113.1 Diffraction Data Collection

The crystals were individually harvested from their trays and transferred to a cryoprotectant consisting of 80% well solution, 10% glycerol, and 10% ethylene glycol. After about 30 seconds the crystal was collected and frozen in a stream of nitrogen gas that had been cooled to 95 degrees Kelvin. The crystals were then transferred in liquid nitrogen to the Advanced Photon Source (Argonne National Laboratory) where a two wavelength MAD experiment was collected, a peak wavelength and a high energy remote wavelength.

11.3.2 Structure Determination

This data was not sufficient on its own to solve the structure. However, the LuxS model from the P2₁ structure from D. radiodurans was used along with the molecular replacement program EPMR to give a solution using the high energy remote data calculated to average anomalous differences. The resulting map was viewed in the program O and found to be of excellent quality with essentially all of both of the proteins in the asymmetric unit, main chain and sidechains, easily visible. This map was modeled using O to give the position of nearly all of the residues: residues 7 through 158. The model was refined using the program CNX.

11.3.3 Structure Analyses

The stereochemical quality of the atomic model was monitored using PROCHECK (Laskowski et al., 1993, “PROCHECK: a program to check the stereochemical quality of LuxS structures,” J. Appl. Cryst. 26:283-291). As defined in PROCHECK, 91.8% of the residues in the model of D. radiodurans C2 LuxS have main chain torsion angles in the most favored Ramachandran regions. No residues fell in the disallowed region, and no residues fell in the generously allowed regions. The overall G-factor score is 0.30.

### 12. EXAMPLE 6

### Preparation of P1 Crystal of D. radiodurans LuxS Protein

D. radiodurans LuxS was expressed, purified, and crystallized essentially as in Example 5, with the exception that the crystallization was carried out at 9° C. instead of 4° C. and that selenomethionine was added during crystallization.

12.1 Structure Determination

This data was not sufficient on its own to solve the structure. However, the LuxS model from the P2₁ structure from D. radiodurans was used along with the molecular replacement program EPMR to give a solution using the high energy remote data calculated to average anomalous differences. The resulting map was viewed in the program O and found to be of excellent quality with essentially all of both of the proteins in the asymmetric unit, main chain and sidechains, easily visible. This map was modeled using O to give the position of nearly all of the residues. The model was refined using the program CNX.

12.2 Structure Analyses

The stereochemical quality of the atomic model was monitored using PROCHECK (Laskowski et al., 1993, “PROCHECK: a program to check the stereochemical quality of LuxS structures,” J. Appl. Cryst. 26:283-291). As defined in PROCHECK, 93.3% of the residues in the model of D. radiodurans P1 LuxS have main chain torsion angles in the most favored Ramachandran regions. No residues fell in the disallowed region, and no residues fell in the generously allowed regions. The overall G-factor score is 0.1.

TABLE 2
Data coliection and refinement statistics

| | | | Completeness (%) | Rsym (%) | MAD |
|---|---|---|---|---|---|
| | Wavelength (Å) | Resolution (Å) | Overall/Outer Shell | Overall/Outer Shell | F.O.M. |
| LuxS MAD data | | | | | |
| collection | | | | | |
| H. pylon | 0.9795 (L1) | 37.0-2.38 | 100.0/100.0 | 5.6/19.8 | 0.66 |
| | 0.9641 (L2) | 37.0-2.38 | 100.0/100.0 | 5.5/26.0 | |
| H. influenzae | 0.9795 (L1) | 32.0-2.10 | 99.6/98.6 | 6.4/18.4 | 0.68 |
| | 0.9641 (L2) | 32.0-2.10 | 99.4/98.1 | 5.7/20.3 | |
| D. radiodurans, | 0.9795 (L1) | 23.0-1.80 | 86.0/100.0 | 7.0/15.3 | 0.60 |
| space group P2₁ | 0.9641 (L2) | 23.0-1.80 | 85.6/99.2 | 5.8/19.6 | |

| | | | Completeness (%) | Rsym (%) | | |
|---|---|---|---|---|---|---|
| | Waveleneth (Å) | Resolution (Å) | Overall/Outer Shell | Overall/Outer Shell | R | C.C. |
| LuxS MR data | | | | | | |
| collection | | | | | | |
| D. radiodurans, C2 | 0.9641 | 35.0-2.10 | 98.1/100.0 | 5.3/7.4 | 0.39 | 0.54 |
| with methionine | | | | | | |
| D. radiodurans, C2 | 1.5418 | 39.0-2.00 | 99.9/98.5 | 4.9/7.1 | 0.40 | 0.58 |
| with no methionine | | | | | | |
| D. radiodurans, | 0.9641 | 48.0-1.65 | | | | |
| P1 with | | | | | | |
| selenomethionine | | | | | | |

| | | | | | RMSD | RMSD | |
|---|---|---|---|---|---|---|---|
| | | | | | Bond | Bond | |
| | | | | | Length | Angles | values (Å²) |
| | Resolution (Å) | R | Rfree | Waters | (Å) | (°) | (mol A/mol B) |
| Refinement | | | | | | | |
| Statistics | | | | | | | |
| H. pylon | 35.0-2.38 | 0.219 | 0.264 | 21 | 0.009 | 1.5 | 34.9/33.7 |
| H. influenzae | 30.0-2.10 | 0.213 | 0.238 | 57 | 0.008 | 1.4 | 26.7/25.9 |
| D. radiodurans, P21 | 20.0-1.80 | 0.206 | 0.240 | 120 | 0.009 | 1.5 | 18.0/18.5 |
| D. radiodurans, C2 | 35.0-2.10 | 0.196 | 0.243 | 46 | 0.007 | 1.4 | 18.6 |
| with methionine | | | | | | | |
| D. radiodurans, C2 | 35.0-2.00 | 0.182 | 0.236 | 80 | 0.009 | 1.5 | 11.3 |
| with no methionine | | | | | | | |
| D. radiodurans, | 36.0-1.65 | 0.188 | 0.209 | 254 | 0.014 | 2.3 | 15.9 |
| P1 with | | | | | | | |
| selenomethionine | | | | | | | |

Rsym = SₕₖₗSᵢ|Iᵢ(hkl)-<I(hkl)>|/SₕₖₗSᵢIᵢ(hkl), where Iᵢ is the intensity of the observation and <I> is the mean intensity of the reflection.
F.O.M is the Figure of merit = <|SP(a)e^{ia}/S|P(a)|>, where a is the phase and P(a) is the phase probability distribution.
R = S∥F_{o|-|F}_{c}∥/S|Fₒ|, where Fₒ and F_{c} are the observed and calculated structure-factor amplitudes.
Rfree is the same as R except calculated for 10% of the data randomly omitted from the refinement.
C.C. is the correlation coefficient = <(|Fₒ|-<|Fₒ|>)(|F_{c}|-<|F_{c}|)>/sqrt[<(|Fₒ|-<|Fₒ|>)²><(|F_{c}|-<|F_{c}|>)²>].
RMSD is root-mean-square deviation.
Average B-values are reported for all non-hydrogen atoms and are separated into A and B molecules in the asymmetric unit when appropriate.

Table 7, Table 8, Table 9, Table 10 , Table 11, and Table 12 provide the atomic structure coordinates of LuxS. In Tables 7 through 12, coordinates for the two LuxS molecules comprising the asymmetric unit are provided, one labeled molecule A and the other molecule B.

The following abbreviations are used in Table 7, Table 8, Table 9, Table 10, Table 11, and Table 12:

“Atom Type” refers to the element whose coordinates are provided. The first letter in the column defines the element.

“A.A.” refers to amino acid.

“X Y and Z” provide the Cartesian coordinates of the element.

“B” is a thermal factor that measures movement of the atom around its atomic center.

“OCC” refers to occupancy, and represents the percentage of time the atom type occupies the particular coordinate. OCC values range from 0 to 1, with 1 being 100%.

“PRT1” or “PRT2” relate to occupancy, with PRT1 designating the coordinates of the atom when in the first conformation and PRT2 designating the coordinates of the atom when in the second or alternate conformation.

Where a particular amino acid residue and number is referred to, that residue number refers to the numbering schema used in Tables 7 through 10, and where one of ordinary skill in the art would understand a corresponding amino acid to be located. For Tables 11 and 12, which follow a slightly different numbering schema, the following conversions apply:

Met84 (Table 7)=Met 80 (Tables 11 and 12); Thr88 (Table 7)=Thr 84 (Tables 11 and 12); Arg87 (Table 7)=Arg83 (Tables 11 and 12); hydrophobic 129 (Table 7)=hydrophobic 123 (Tables 11 and 12); Gly132 (Table 7)=Gly126 (Tables 11 and 12); hydrophobic 134 (Table 7)=hydrophobic 128 (Tables 11 and 12); Cys86 (Table 7)=Cys82 (Tables 11 and 12); Tyr91 (Table 7)=Tyr87 (Table 11 and 12); Glu128 (Table 7)=Glu122 (Tables 11 and 12).

Structure coordinates for LuxS molecules according to Tables 7-11, may be modified by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates and any combination of the above.

### 13. FORMULATION AND ADMINISTRATION

Pharmaceutical compositions comprising LuxS affectors, preferably inhibitors, are useful, for example, as antimicrobial agents and for aiding the treatment of cystic fibrosis. While these compounds will typically be used in therapy for human patients, they may also be used in veterinary medicine to treat similar or identical diseases, and may also be used as plant herbicides and antimicrobials. Pharmaceutical compositions containing LuxS affecters may also be used to modify the activity of human homologs of LuxS.

In therapeutic and/or diagnostic applications, the compounds of the invention can be formulated for a variety of modes of administration, including systemic and topical or localized administration. Techniques and formulations generally may be found in Remington's Pharmaceutical Sciences, 18^{th} ed., Mack Publishing Co. (1990).

The compounds according to the invention are effective over a wide dosage range. For example, in the treatment of adult humans, dosages from 0.01 to 1000 mg, preferably from 0.5 to 100 mg, and more preferably from 1 to 50 mg per day, more preferably from 5 to 40 mg per day may be used. A most preferable dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington's Pharmaceutical Sciences; (18th ed.), Mack Publishing Co., Easton, Pa. (1990). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained- low release form as is known to those skilled in the art. Techniques for formulation and administration may be found in Remington's Pharmaceutical Sciences; (18th ed.), Mack Publishing Co., Easton, Pa. (1990). Suitable routes may include oral, buccal, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, preferably in physiologically compatible buffers such as Hank's solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

The present invention is not to be limited in scope by the exemplified embodiments, which are intended as illustrations of single aspects of the invention. Indeed, various modifications of the invention in addition to those described herein will become apparent to those having skill in the art from the foregoing description and accompanying drawings. Such modifications are intended to fall with in the scope of the appended claims.

All references cited herein are hereby incorporated by reference in their entirety.

1. A method of identifying a candidate binding compound capable of binding to the active site of LuxS protein, said method comprising:
a) introducing into a computer program information derived from structural coordinates defining an active site conformation of a LuxS protein molecule based upon three-dimensional structure determination comprising an active site formed by at least the interaction of amino acids Glu, Arg, Ile, and Asp, wherein said program utilizes or displays the three-dimensional structure thereof;
b) generating a three-dimensional representation of the active site cavity of said LuxS protein in said computer program;
c) superimposing a model of the binding test compound on the model of said active site of said LuxS protein; and
d) assessing whether said test compound model fits spatially into the active site of said LuxS protein.

2. The method of claim 1, further comprising the steps of:
a) incorporating said binding test compound in a binding assay for a protein characterized by said active site of said LuxS protein; and
b) determining whether said test compound binds to said protein in said assay.

3. The method of claim 1, wherein said active site further comprises amino acids corresponding to Ala, His, Tyr, Ser, Phe and Leu.

4. The method of claim 3, wherein said active site further comprises amino acids corresponding to Met, Cys, and Thr.

5. The method of claim 1, wherein said amino acids correspond to Glu60, Arg68, Ile81, and Asp80.

6. The method of claim 1, wherein said active site further comprises amino acids corresponding to Ala64, His61, Tyr91, Ser9, Phe10 and Leu7.

7. The method of claim 5, wherein said active site further comprises amino acids corresponding to His14, Arg23, Asp40, Arg42, Met84, Cys86, and Thr88.

8. The method of any of claims 1-7, wherein the structural coordinates correspond to the liganded or unliganded LuxS protein.

9. The method of claim 8, wherein said active site is defined by the structural coordinates of Table 7, Table 8, Table 9, Table 10, Table 11 or Table 12.

10. The method of claim 1, wherein said binding compound is a LuxS inhibitor.

11. The method of claim 10, further comprising the steps of:
e) incorporating said inhibitor test compound in an activity assay for a protein characterized by said active site of said LuxS protein; and
f) determining whether said test compound inhibits said protein activity in said assay.

12. The method of claim 11, wherein said activity assay comprises a LuxS protein inhibition assay.

13. The method of claim 1, wherein the LuxS is H. pylori LuxS, H. influenzae LuxS, or D. radiodurans LuxS.

14. A method of selecting at least one compound that potentially binds to LuxS protein, comprising,
(a) constructing a three-dimensional structure of LuxS protein having structure coordinates selected from the group consisting of the structure coordinates of the crystals of claims 77-89, the structure coordinates of Table 7, Table 8, Table 9, Table 10, Table 11, or Table 12, and the structure coordinates of a protein having a root mean square deviation of the alpha carbon atoms, of less than 2.0 Å when compared to the structure coordinates of Table 7, Table 8, Table 9, Table 10, Table 11, or Table 12, or a portion thereof; and
(b) selecting at least one compound which potentially binds LuxS protein;
wherein said selecting is performed with the aid of the structure constructed in step (a).

15. A method of identifying at least one compound that potentially binds to LuxS protein, comprising,
a. constructing a three-dimensional structure of a protein molecule comprising a LuxS protein binding pocket, wherein said binding pocket is defined by the structure coordinates of at least three amino acids selected from the group consisting of Glu60, Arg68, Ile81, and Asp80, Ala64, His61, Tyr91, Ser9, Phe10 and Leu7, His14, Arg23, Asp40, Arg42, Met84, Cys86, and Thr88, or a protein molecule comprising a LuxS protein binding pocket homolog, wherein the root mean square deviation of the structure coordinates of the backbone atoms of the amino acids of said binding pocket and said binding pocket homolog is less than 2.0 Å.
b. computationally screening a plurality of compounds using the structure constructed in step (a), and identifying at least one compound that computationally binds to said structure.

16. The method of claim 15, wherein said binding pocket comprises Glu60, Arg68, Ile81, and Asp80.

17. The method of claim 16, wherein said binding pocket further comprises Ala64, His61, Tyr91, Ser9, Phe10 and Leu7.

18. The method of claim 17, wherein said binding pocket further comprises His14, Arg23, Asp40, Arg42, Met84, Cys86, and Thr88.

19. The method of claim 15, wherein said binding compound is a LuxS inhibitor.

20. The method of claim 15, wherein the LuxS is H. pylori LuxS, H. influenze LuxS, or D. radiodurans LuxS.

21. A method of identifying a compound that binds LuxS protein, comprising computationally screening a three-dimensional structural representation of a molecule comprising a LuxS protein binding pocket, wherein said binding pocket comprises at least three amino acids selected from the group consisting of Glu60, Arg68, Ile81, and Asp80, Ala64, His61, Tyr91, Ser9, Phe10 and Leu7, His14, Arg23, Asp40, Arg42, Met84, Cys86, and Thr88, having structure coordinates according to Table 7, Table 8, Table 9, Table 10, Table 11, or Table 12, or a molecule comprising a binding pocket homolog that has a root mean square deviation of the structure coordinates of the backbone atoms of said amino acids of less than 2.0 Å, with a plurality of compounds, and identifying those that bind.

22. The method of claim 21, wherein said amino acids comprise Glu60, Arg68, Ile81, and Asp80.

23. The method of claim 22, wherein said amino acids further comprise Ala64, His61, Tyr91, Ser9, Phe10 and Leu7.

24. The method of claim 23, wherein said amino acids further comprise His14, Arg23, Asp40, Arg42, Met84, Cys86, and Thr88.

25. The method of claim 21, wherein said binding compound is a LuxS inhibitor.

26. The method of claim 21, wherein the LuxS is H. pylori LuxS, H. influenze LuxS, or D. radiodurans LuxS.

27. The method of claim 21, wherein determining whether the compound potentially binds to said molecule comprises performing a fitting operation between the compound and a binding pocket of the molecule or molecular complex; and computationally analyzing the results of the fitting operation to quantify the association between, or the interference with, the compound and the binding pocket.

28. The method of claim 21, further comprising screening a library of compounds.

29. A method for designing a compound that modulates LuxS protein activity, comprising:
(a) providing a computer modeling program with a set of structure coordinates, or a three dimensional conformation derived therefrom, for a molecule that comprises a binding pocket having the structure coordinates of the binding pocket of LuxS protein, or a binding pocket homolog;
(b) computationally building a chemical entity represented by set of structure coordinates; and
(c) and determining whether the chemical entity is a modulator expected to bind to or interfere with the molecule wherein binding to the molecule is indicative of potential inhibition of LuxS protein activity.

30. The method of claim 29, wherein said binding pocket comprises at least three amino acid residues selected from the group consisting of Glu60, Arg68, Ile81, and Asp80, Ala64, His61, Tyr91, Ser9, Phe10 and Leu7, His14, Arg23, Asp40, Arg42, Met84, Cys86, and Thr88.

31. The method of claim 30, wherein said binding pocket comprises amino acid residues Glu60, Arg68, Ile81, and Asp80.

32. The method of claim 31, wherein said amino acids further comprise Ala64, His61, Tyr91, Ser9, Phe10 and Leu7.

33. The method of claim 32, wherein said amino acids further comprise His14, Arg23, Asp40, Arg42, Met84, Cys86, and Thr88.

34. The method of claim 29, wherein the LuxS is H. pylori LuxS, H. influenze LuxS, or D. radiodurans LuxS.

35. A method of identifying a candidate binding compound capable of binding to an accessory binding site of LuxS protein, said method comprising:
a) introducing into a computer program information derived from structural coordinates defining an accessory binding site conformation of a LuxS protein molecule based upon three-dimensional structure determination comprising an accessory binding site formed by at least the interaction of amino acids His57, His61, Cys131, and His137, wherein said program utilizes or displays the three-dimensional structure thereof;
b) generating a three-dimensional representation of the accessory binding site cavity of said LuxS protein in said computer program;
c) superimposing a model of the binding test compound on the model of said accessory binding site of said LuxS protein; and
d) assessing whether said test compound model fits spatially into the accessory binding site of said LuxS protein.

36. The method of claim 35, further comprising the steps of:
e) incorporating said binding test compound in a binding assay for a protein characterized by said accessory binding site of said LuxS protein; and
f) determining whether said test compound binds to said protein in said assay.

37. The method of claim 35, wherein said binding compound is a LuxS inhibitor.

38. The method of claim 37, further comprising the steps of:
a) incorporating said inhibitor test compound in an activity assay for a protein characterized by said active site of said LuxS protein; and
b) determining whether said test compound inhibits said protein activity in said assay.

39. The method of claim 35, wherein the LuxS is H. Pylori LuxS, H. influenzae LuxS, or D. radiodurans.

40. The method of claim 36, wherein the LuxS is H. Pylori LuxS, H. influenzae LuxS, or D. radiodurans.

41. The method of any of claims 1-10, 12-35 and 40, further comprising supplying or synthesizing the test compound, then assaying the compound to determine whether it modulates LuxS protein activity.

42. A compound identified, designed or made according to the method of any of claims 1-41.

43. A pharmaceutical composition comprising a compound of claim 42, or a salt thereof, and a pharmaceutically acceptable carrier.

44. A method of modulating LuxS protein activity, comprising contacting said LuxS protein with a compound of claim 42.

45. A method for obtaining structural information about a molecule or a molecular complex of unknown structure comprising:
(a) crystallizing the molecule or molecular complex;
(b) generating an x-ray diffraction pattern from the crystallized molecule or molecular complex; and
(c) using a molecular replacement method to interpret the structure of said molecule; wherein said molecular replacement method uses the structure coordinates of Table 7, Table 8, Table 9, Table 10, Table 11, or Table 12, or a subset thereof comprising the binding pocket, the structure coordinates of the binding pocket of Table 7, Table 8, Table 9, Table 10, Table 11, or Table 12, or structure coordinates having a root mean square deviation for the alpha-carbon atoms atoms of said structure coordinates of less than 2.0 Å.

46. A method for homology modeling a LuxS protein homolog comprising:
(a) aligning the amino acid sequence of a LuxS protein homolog with an amino acid sequence of LuxS protein;
(b) incorporating the sequence of the LuxS protein homolog into a model of the structure of LuxS protein, wherein said model has the same structure coordinates as the structure coordinates of Table 7, Table 8, Table 9, Table 10, Table 11, or Table 12, or wherein the structure coordinates of said model's alpha-carbon atoms have a root mean square deviation from the structure coordinates of Table 7, Table 8, Table 9, Table 10, Table 11, or Table 12, of less than 2.0 Å yield a preliminary model of said homolog;
(c) subjecting the preliminary model to energy minimization to yield an energy minimized model; and
(d) remodeling regions of the energy minimized model where stereochemistry restraints are violated to yield a final model of said homolog.

47. The method of claim 46, wherein the LuxS is H. pylori LuxS, H. influenze LuxS, or D. radiodurans LuxS.

48. A crystal comprising LuxS protein, or a functional LuxS protein subunit, in crystalline form.

49. The crystal of claim 48, which is diffraction quality.

50. The crystal of claim 48, which is an apo-crystal

51. The crystal of claim 48, which is a native crystal.

52. The crystal of claim 48, wherein the LuxS is H. pylori LuxS, H. influenze LuxS, or D. radiodurans LuxS.

53. The crystal of claim 48, which is a heavy-atom derivative crystal.

54. The crystal of claim 48, in which LuxS protein is a mutant.

55. The crystal of claim 54, in which the mutant is a selenomethionine or selenocysteine mutant.

56. The crystal of claim 54, in which the mutant is a conservative mutant.

57. The crystal of claim 54, in which the mutant is a truncated or extended mutant.

58. The crystal of claim 48, which is characterized by a set of structure coordinates that is substantially similar to the set of structure coordinates of Table 7, Table 8, Table 9, Table 10, Table 11, or Table 12.

59. A crystal comprising a homolog of LuxS protein, wherein said homolog has a root mean square deviation of the alpha-carbon atoms of less than 2.0 Å.

60. The crystal of claim 48, which is produced by a method comprising the steps of:
(a) mixing a volume of a solution comprising the LuxS protein with a volume of a reservoir solution comprising a precipitant; and
(b) incubating the mixture obtained in step (a) over the reservoir solution in a closed container, under conditions suitable for crystallization until the crystal forms.

61. A method of making the crystal of claim 48, comprising:
(a) mixing a volume of a solution comprising the LuxS protein with a volume of a reservoir solution comprising a precipitant; and
(b) incubating the mixture obtained in step (a) over the reservoir solution in a closed container, under conditions suitable for crystallization until the crystal forms.

62. A method for determining the three-dimensional structure of a LuxS protein crystal, comprising the steps of
(a) providing a crystal of any of claims 48-61; and
(b) analyzing said crystal by x-ray diffraction to determine said three-dimensional structure.

63. A machine-readable medium embedded with information that corresponds to a three-dimensional structural representation of a crystal of any of claims 48-61.

64. The machine-readable medium of claim 63, in which the information comprises the molecular structure coordinates that correspond to the three-dimensional structural representation.

65. A machine-readable medium embedded with the molecular structure coordinates of Table 7, Table 8, Table 9, Table 10, Table 11, or Table 12, or at least 50% of the coordinates thereof.

66. A machine-readable medium embedded with the molecular structure coordinates of Table 7, Table 8, Table 9, Table 10, Table 11, or Table 12, or at least 80% of the coordinates thereof.

67. A machine-readable medium embedded with the molecular structure coordinates of a protein molecule comprising a LuxS protein binding pocket, wherein said binding pocket comprises at least three amino acids selected from the group consisting of Glu60, Arg68, Ile81, and Asp80, Ala64, His61, Tyr91, Ser9, Phe10 and Leu7, His14, Arg23, Asp40, Arg42, Met84, Cys86, and Thr88, having the structure coordinates of Table 7, Table 8, Table 9, Table 10, Table 11, or Table 12, or by the structure coordinates of a binding pocket homolog, wherein said the root mean square deviation of the backbone atoms of the amino acid residues of said binding pocket and said binding pocket homolog is less than 2.0 Å.

68. The machine-readable medium of claim 67, wherein the LuxS is H. pylori LuxS, H. influenze LuxS, or D. radiodurans LuxS.

69. The machine-readable medium of claim 67, wherein said binding pocket comprises Glu60, Arg68, Ile81, and Asp80.

70. The machine readable medium of claim 69, wherein said binding pocket further comprises Ala64, His61, Tyr91, Ser9, Phe10 and Leu7.

71. The machine readable medium of claim 70, wherein said binding pocket further comprises His14, Arg23, Asp40, Arg42, Met84, Cys86, and Thr88.

72. A method of producing a mutant of LuxS protein, having an altered property relative to LuxS protein, comprising,
(a) constructing a three-dimensional structure of LuxS protein having structure coordinates selected from the group consisting of the structure coordinates of the crystals of claims 48-61, the structure coordinates of Table 7, Table 8, Table 9, Table 10, Table 11, or Table 12, and the structure coordinates of a protein having a root mean square deviation of the alpha carbon atoms of said protein of less than 2.0 A when compared to the structure coordinates of Table 7, Table 8, Table 9, Table 10, Table 11, or Table 12;
(b) using modeling methods to identify in the three-dimensional structure at least one structural part of the LuxS protein molecule wherein an alteration in said structural part is predicted to result in said altered property;
(c) providing a nucleic acid molecule having a modified sequence that encodes a deletion, insertion, or substitution of one or more amino acids at a position corresponding to said structural part; and
(d) expressing said nucleic acid molecule to produce said mutant;
wherein said mutant has at least one altered property relative to the parent.

73. The method of claim 72, wherein the LuxS is H. pylori LuxS, H. influenze LuxS, or D. radiodurans LuxS.

74. The method of claim 72, wherein said mutant has altered LuxS protein activity.

75. The method of claim 72, wherein said mutant has altered binding activity.

76. The method of claim 72, wherein said mutant has altered immunogenicity.

77. The method of claim 72, wherein said mutant has at least one epitope that is altered.


SEQUENCE LISTING (13 sequences)

SEQ ID NO: 1 - protein, 152 aa, H. Pylori
MKMNVESFNLDHTKVKAPYVRIADRKKGVNGDLIVKYDVRFKQPNRDHMDMPSLHSLEHLVAEIIRNHANYVVDWSPMGCQTGFYLTVLNHDNYTEILEVLEKTMQDVLKAKEVPASNEKQCGWAANHTLEGAQNLARAFLDKRAEWSEVGV

SEQ ID NO: 2 - protein, 167 aa, H. Influenzae
MPLLDSFKVDHTKMNAPAVRIAKTMLTPKGDNITVFDLRFCIPNKEILSPKGIHTLEHLFAGFMRDHLNGDSIEIIDISPMGCRTGFYMSLIGTPNEQKVSEAWLASMQDVLGVQDQASIPELNIYQCGSYTEHSLEDAHEIAKNVIARGIGVNKNEDLSLDNSLLK

SEQ ID NO: 3 - protein, 158 aa, D. Radiodurans
MPDMANVESFDLDHTKVKAPYVRLAGVKTTPKGDQISKYDLRFLQPNQGAIDPAAIHTLEHLLAGYMRDHLEGVVDVSPMGCRTGMYMAVIGEPDEQGVMKAFEAALKDTAGHDQPIPGVSELECGNYRDHDLAAARQHARDVLDQGLKVQETILLER

SEQ ID NO: 4 - protein, 164 aa, C. Jejuni
MPLLDSFKVDHTKMPAPAVRLAKVMKTPKGDDISVFDLRFCIPNKDIMSEKGTHTLEHLFAGFMRDHLNSNSVEIIDISPMGCRTGFYMSLIGTPDEKSIAKAWEAAMKDVLSVSDQSKIPELNIYQCGTCAMHSLDEAKQIAQKVLNLGISIINNKRLKLENA

SEQ ID NO: 5 - protein, 157 aa, B. Burgdorferi
MKKITSFTIDHTKLNPGIYVSRKDTFENVIFTTIDIRIKAPNIEPIIENAAIHTIEHIGATLLRNNEVWTEKIVYFGPMGCRTGFYLIIFGDYESKDLVDLVSWLFSEIVNFSEPIPGASDKECGNYKEHNLDMAKYESSKYLQILNNIKEENLKYP

SEQ ID NO: 6 - protein, 151 aa, C. Perfringens
MVKVESFELDHTKVKAPYVRKAGIKIGPKGDIVSKFDLRFVQPNKELLSDKGMHTLEHFLAGFMREKLDDVIDISPMGCKTGFYLTSFGDIDVKDIIEALEYSLSKVLEQEEIPAANELQCGSAKLHSLELAKSHAKQVLENGISDKFYVE

SEQ ID NO: 7 - protein, 168 aa, N. Meningitidis
MPLLDSFKVDHTRMHAPAVRVAKTMTTPKGDTITVFDLRFCVPNKEILPEKGIHTLEHLFAGFMRDHLNGNGVEIIDISPMGCRTGFYMSLIGTPSEQQVADAWLASMQDVLNVKDQSKIPELNEYQCGTYQMHSLAEAQQIAQNVLARKVAVNKNEELTLDEGLLNA

SEQ ID NO: 8 - protein, 171 aa, S. Typhimurium
MPLLDSFAVDHTRMQAPAVRVAKTMNTPHGDAITVFDLRFCIPNKEVMPEKGIHTLEHLFAGFMRDHLNGNGVEIIDISPMGCRTGFYMSLIGTPDEQRVADAWKAAMADVLKVQDQNQIPELNVYQCGTYQMHSLSEAQDIARHILERDVRVNSNKELALPKEKLQELHI

SEQ ID NO: 9 - protein, 172 aa, V. Harveyi
MPLLDSFTVDHTRMNAPAVRVAKTMQTPKGDTITVFDLRFTAPNKDILSEKGIHTLEHLYAGFMRNHLNGDSVEIIDISPMGCRTGFYMSLIGTPSEQQVADAWIAAMEDVLKVENQNKIPELNEYQCGTAAMHSLDEAKQIAKNILEVGVAVNKNDELALPESMLRELRID

SEQ ID NO: 10 - protein, 171 aa, E. Coli
MPLLDSFTVDHTRMEAPAVRVAKTMNTPHGDAITVFDLRFCVPNKEVMPERGIHTLEHLFAGFMRNHLNGNGVEIIDISPMGCRTGFYMSLIGTPDEQRVADAWKAAMEDVLKVQDQNQIPELNVYQCGTYQMHSLQEAQDIARSILERDVRINSNEELALPKEKLQELHI

SEQ ID NO: 11 - protein, 172 aa, V. Cholera
MPLLDSFTVDHTRMNAPAVRVAKTMQTPKGDTITVFDLRTMQPKDILSERGAIHTLEHYLAFYMRNHLNGSQVEIIDISPMGCRTGFYMSLIGAPTEQQVAQAWLAAMQDVLKVESQEQIPELNEYQCGTAAMHSLEEAKAIAKNVIAAGISVNRNDELALPESMLNELKVH

SEQ ID NO: 12 - protein, 231 aa, B. Subtilis
MPSVESFELDHNAVVAPYVRHCGVHKVGTDGVVNKFDIRFCQPNKQAMKPDTIHTLEHLLAFTIRSHAEKYDHFDIIDISPMGCQTGYYLVVSGEPTSAEIVDLLEDTMKEAVEITEIPAANEKQCGQAKLHDLEGAKRLMRFWLSQDKEELIKVFGQTGFYLIMSGKPTVEEIIDVLEQTMKYSLELEEVPAANEKQCGQAKLHDLDGAKKLMTYWLSHEKDSLTKVFES

SEQ ID NO: 13 - protein, 84 aa, B. Halodurans
MPTVESFELDHTIVKAPFVRPCGTHKVGTNGEVNKFDIRFFQPNKQAMKPDVIHTLEHLLALNIRKFAEAYDHFDVIDLSPMGC